(19) United States
(12) Patent Application Publication
(10) Pub. No.: US 2019/0262321 A1
(43) Pub. Date: Aug. 29, 2019

(54) METHODS OF TREATING DIABETIC NEPHROPATHY USING HPTPB INHIBITORS

(71) Applicant: Aerpio Therapeutics, Inc., Cincinnati, OH (US)

(72) Inventors: Kevin PETERS, Cincinnati, OH (US); Mitchell BRIGELL, Belmont, MA (US); Stephen PAKOLA, Irvington, NY (US); Robert Shalwitz, Bexley, OH (US)

(21) Appl. No.: 16/284,349

(22) Filed: Feb. 25, 2019

Related U.S. Application Data
(60) Provisional application No. 62/790,646, filed on Jan. 10, 2019.
(60) Provisional application No. 62/635,102, filed on Feb. 26, 2018.

(51) Int. Cl. A61K 31/427; A61P 3/00; A61K 31/16; A61P 13/12; A61K 31/137

(52) U.S. Cl. CPC A61K 31/427; A61P 3/00; A61K 31/137; A61P 13/12; A61K 31/16

(57) ABSTRACT

Disclosed herein are compounds effective for activation of Tie-2 and inhibition of HPTP-beta. The compounds can provide effective therapy for conditions associated with diabetic nephropathy, for example, diabetic nephropathy resulting from hyperglycemia, kidney hyperfiltration, renal injury, glycation products, and cytokine activation.

### CROSS REFERENCE

This application claims the benefit of U.S. Provisional Application No. 62/635,102, filed Feb. 26, 2018, and U.S. Provisional Application No. 62/790,646, filed Jan. 10, 2019, each of which is incorporated herein by reference in its entirety.

### BACKGROUND

Diabetic nephropathy is a significant pathology associated with diabetes. Diabetic nephropathy can result from hyperglycemia, kidney hyperfiltration, renal injury, glycation products, and cytokine activation. Loss of kidney function due to diabetic nephropathy can lead to low serum albumin, edema, and end-stage kidney disease.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the disclosure provides a method of treating nephropathy in a subject in need thereof, the method comprising administering to the subject a therapeutically-effective amount of a compound that activates Tie-2 or a pharmaceutically-acceptable salt thereof.

In some embodiments, the disclosure provides a method of treating neuropathy in a subject in need thereof, the method comprising administering to the subject a therapeutically-effective amount of a compound that inhibits HPTPβ or a pharmaceutically-acceptable salt thereof.

### BRIEF DESCRIPTION OF THE FIGURES

FIG. 1 shows an experimental set-up of a study testing the effect of compound AA34.

FIG. 2 shows the results of the renal function assessment study as the % change from baseline to end of treatment (three months) in geometric mean +95% confidence interval in patients with albuminuria at baseline.

FIG. 3 shows the expression of VE-PTP in kidney tissue endothelium.

FIG. 4 PANEL A shows that LPS caused an abrupt rise in BUN, which was statistically reduced in mice treated with phosphatase inhibitor compound AA34 (*p=0.002). PANEL B shows a histological image of kidney tissue obtained from a mouse treated with LPS+vehicle. PANEL C shows a histological image of kidney tissue obtained from a mouse treated with LPS+phosphatase inhibitor compound AA34, that shows reduced kidney tubule damage compared to LPS+vehicle.

FIG. 5 shows that the group treated with LPS+Compound AA34 exhibited increased levels of tyrosine phosphorylated Tie2 compared to the group treated with LPS+vehicle.

FIG. 6 PANEL A shows DNA laddering used to detect apoptosis in kidney homogenate; each lane represents a different animal. DNA laddering was decreased in animals receiving AA34 compared to animals receiving vehicle. PANEL B shows TUNEL staining for apoptotic nuclei (dark black stain circled) in kidney tissue obtained from animals treated with LPS+vehicle. PANEL C shows decreased TUNEL staining in kidney tissue obtained from animals treated with LPS+Compound AA34.

FIG. 7 PANEL A shows that treatment with compound AA34 significantly reduced renal neutrophil infiltration 24 h after LPS injection. PANEL B shows LPS-induced renal neutrophil infiltration in animals treated with LPS+vehicle. PANEL C shows reduced LPS-induced renal neutrophil infiltration in animals treated with LPS+Compound AA34 compared to animals treated with vehicle.

FIG. 8 PANEL A shows that phosphatase inhibition by compound AA34 reduced renal mRNA expression of TNFR1 and PAI-1. PANEL B shows that phosphatase inhibition by compound AA34 reduced renal mRNA expression of tissue factor and iNOS.

FIG. 9 PANEL A shows a baseline image of a mouse injected with 70 kDa fluorescent fixable dextrans. PANEL B shows a fluorescence image of a mouse injected with LPS and subsequently injected with 70 kDa fluorescent fixable dextrans. PANEL C shows a fluorescence image of a mouse injected with LPS+AA34, and subsequently injected with 70 kDa fluorescent fixable dextrans. PANEL D shows a baseline image of a mouse injected with 500 kDa fluorescent fixable dextrans. PANEL E shows a fluorescence image of a mouse injected with LPS and subsequently injected with 500 kDa fluorescent fixable dextrans. PANEL F shows a fluorescence image of a mouse injected with LPS+AA34, and subsequently injected with 500 kDa fluorescent fixable dextrans. PANEL G shows a baseline image of a mouse injected with tomato lectin. PANEL H shows a fluorescence image of a mouse injected with LPS and subsequently injected with tomato lectin. PANEL I shows a fluorescence image of a mouse injected with LPS+AA34, and subsequently injected with tomato lectin.

### DETAILED DESCRIPTION

Described herein are therapies using an HPTPβ inhibitor for treatment of diabetic nephropathy. An HPTPβ inhibitor of the disclosure can bind to HPTPβ, thereby activating Tie-2 signaling by promoting protein phosphorylation, such as phosphorylation of the Tie-2 protein.

Tie-2 (tyrosine kinase with immunoglobulin and epidermal growth factor homology domains 2) is a membrane receptor tyrosine kinase expressed primarily in vascular endothelial cells and a subset of hematopoietic stem cells (HSCs) and macrophages. The principle regulators of Tie-2 phosphorylation are angiopoietin 1 (Ang-1) and angiopoietin 2 (Ang-2). Ang-1 is an agonist of Tie-2, and binding of Ang-1 to Tie-2 promotes receptor phosphorylation. Ang-2 is a Tie-2 ligand that acts in a context-dependent antagonistic or agonistic manner. Binding of Ang-1 to Tie-2 increases the level of endogenous Tie-2 receptor phosphorylation and initiates downstream AKT signaling. This binding initiates a signaling cascade that can induce distinctive vascular remodeling through highly organized angiogenesis and tightening of the endothelial cell junctions (endothelium cell proximity). Within the vascular endothelium, Ang-1-Tie-2 signaling promotes endothelial cell proximity. In the HSC microenvironment, Ang-1-Tie-2 signaling contributes in a paracrine manner to the long-term repopulation of HSCs.

Under physiological conditions, the extent and duration of Tie-2 phosphorylation is regulated by the human protein tyrosine phosphatase beta (often abbreviated as HPTPβ or HPTP beta), which removes the phosphate from the Tie-2 receptor. By inhibiting HPTPβ, the level of Tie-2 phosphorylation substantially increases, restoring proper endothelial cell proximity and functions. HPTPβ plays a functional role in endothelial cell viability, differentiation, permeability, and interactions with inflammatory and endothelial support cells, such as pericytes, podocytes, and smooth muscle cells. HPTPβ and vascular endothelial protein tyrosine phosphatase (VE-PTP; the mouse orthologue of HPTPβ) are expressed in vascular endothelial cells throughout development and in the adult vasculature. A small molecule of the disclosure can activate Tie-2 downstream signaling by inhibiting HPTPβ/VE-PTP.

A therapy of the disclosure can be used to treat diabetic nephropathy. Diabetic nephropathy is the chronic loss of kidney function in subjects with diabetes mellitus. Diabetic nephropathy can result from hyperglycemia, kidney hyperfiltration, renal injury, glycation products, and cytokine activation. Physiological changes in subjects with diabetes can damage the kidney's glomeruli, the networks of tiny blood vessels in the kidneys, and lead to albumin in the urine (albuminuria). Loss of kidney function due to diabetic nephropathy can lead to low serum albumin, edema, and end-stage kidney disease.

Diabetic nephropathy affects the ability of the kidney to remove waste products and extra fluid from the body. Early stages of diabetic nephropathy can be asymptomatic. Symptoms of later stage diabetic nephropathy include, for example, worsening blood pressure control, protein in the urine, swelling of extremities, increased need to urinate, less need for insulin or diabetes medicine, confusion or difficulty concentrating, loss of appetite, nausea and vomiting, persistent itching, and fatigue. Complications of diabetic nephropathy can develop gradually and include, for example, fluid retention, which can lead to swelling in the extremities, high blood pressure, or fluid in the lungs (pulmonary edema); a sudden rise in potassium levels in the blood (hyperkalemia); heart and blood vessel disease (cardiovascular disease); stroke; damage to the blood vessels of the retina (diabetic retinopathy); anemia; foot sores; erectile dysfunction; diarrhea; and other problems related to damaged nerves and blood vessels; pregnancy complications that carry risks for the mother and the developing fetus; or irreversible damage to the kidneys (end-stage kidney disease), eventually requiring either dialysis or a kidney transplant for survival.

As diabetic nephropathy progresses, a structure in the glomeruli known as the glomerular filtration barrier (GFB) can become increasingly compromised. The GFB is composed of three layers including the fenestrated endothelium, the glomerular basement membrane, and the epithelial podocytes, and is responsible for highly selective filtration of blood entering the kidney's glomeruli. Generally, the GFP only allows the passage of water, small molecules, and very small proteins, not including albumin.

During the course of diabetic nephropathy, three major histologic changes can occur in the glomeruli of subjects afflicted with diabetic nephropathy. First, mesangial expansion can occur due to hyperglycemia, the onset of which can be brought about via increased matrix production or glycation of matrix proteins. Second, thickening of the glomerular basement membrane (GBM) can occur. Finally, glomerular sclerosis can occur due to intraglomerular hypertension, which can be induced by dilatation of the afferent renal artery or from ischemic injury induced by hyaline narrowing of the vessels supplying the glomeruli.

A diagnosis of diabetic nephropathy can be made by measuring the amount of albumin in a subject's urine. The amount of albumin in a subject's urine can be presented as the amount of albumin excreted over a 24-hour period or as a ratio relative to urine creatinine levels. Albuminuria in a subject with diabetic nephropathy can range from about 30 mg/24 h to about or more than 300 mg/24 h albumin excreted over a 24-hour period. In terms of a urine albumin/creatinine ratio (UACR), a diagnosis of albuminuria can be made when the UACR is greater than 30 mg/g (mg of albumin/grams of creatinine).

A therapy of the disclosure can be used to treat acute kidney injury (AKI). AKI is a sudden episode of kidney failure or kidney damage that develops rapidly over a few hours or days. AKI is the leading cause of nephrology consultations and is associated with high mortality rates. The primary causes of AKI include ischemia, hypoxia, and nephrotoxicity. AKI is a common problem among hospitalized patients, and is frequently a consequence of sepsis. The renal endothelium plays a key role in sepsis-induced AKI. Activated Tie-2, expressed mainly in endothelial cell surfaces, can result in protective effects for in sepsis-induced AKI, including downregulation of adhesion molecule expression, inhibition of apoptosis, preservation of barrier function, and regulation of angiogenesis.

AKI is grouped into three primary etiologies: prerenal, renal, and postrenal. Maintaining a normal GFR is dependent on adequate renal perfusion. Prerenal azotemia is characterized by a decrease in GFR due to a decrease in renal perfusion pressure without damage to the renal parenchyma. Because the kidneys receive up to 25% of the cardiac output, inadequate circulation or isolated failure of the intrarenal circulation can have a profound impact on renal perfusion. Causes of prerenal azotemia include hypovolemia resulting from conditions such as hemorrhage, vomiting, diarrhea, poor oral intake, burns, excessive sweating, renal losses, diuresis; impaired cardiac output resulting from congestive heart failure or decreased cardiac output states (e.g. pericardial tamponade, severe pulmonary hypertension); decreased vascular resistance (peripheral vasodilation) resulting from conditions such as sepsis, vasodilator medications, autonomic neuropathy, or anaphylaxis; and renal vasoconstriction from vasoconstrictive medications or conditions such as hypercalcemia.

The normal response of the kidney to prerenal conditions is to concentrate the urine maximally and avidly reabsorb sodium in an effort to maintain/increase intravascular volume and normalize renal perfusion. Therapies that rapidly restore renal perfusion can improve renal function. However, prolonged or profound prerenal azotemia can result in irreversible ischemic damage to the kidney.

Postrenal causes of AKI are characterized by acute obstruction to urinary flow. Urinary tract obstruction increases intratubular pressure and thus decreases GFR. In addition, acute urinary tract obstruction can lead to impaired renal blood flow and inflammatory processes that also contribute to diminished GFR. Obstruction of the urinary tract at any level can cause AKI. Generally, urinary tract obstruction involves both kidneys or a solitary kidney to produce significant renal failure. However, patients with pre-existing renal insufficiencies can develop AKI with obstruction of only one kidney.

Acute tubular necrosis (ATN), or acute renal tubular injury, is a type of AKI that results from damage to the tubules. The two major causes of ATN are ischemic (resulting from severe or protracted decrease in renal perfusion) and nephrotoxic (resulting from a variety of exogenous compounds, for example, aminoglycosides, amphotericin B, cis-platinum, and radiocontrast agents, and endogenous compounds, for example, hemoglobin in hemolysis, myoglobin in rhabdomyolysis, that are toxic or potentially toxic to the kidney).

Classic ATN goes through an oliguric (urine output ≤400 mL/24 hours) phase of 1-2 weeks followed by a non-oliguric (urine output >400 mL/day) phase of 10-14 days with eventual recovery of renal function. However, both prolonged oliguric phases and initial non-oliguric phases are also common.

The initiation phase of ATN occurs when renal blood flow decreases to a level resulting in severe cellular ATP depletion that in turn leads to acute cell injury and dysfunction. Renal tubular epithelial cell injury is a key feature of the Initiation Phase. Renal ischemia in vivo rapidly induces a number of structural and functional alterations in renal proximal tubular epithelial cells that are directly related spatially and temporally with disruption of the normal framework of filamentous actin in the cell. The extent of these alterations depends upon the severity and duration of ischemic injury. Although not lethal to the cell, these alterations disrupt the ability of renal tubular epithelial cells and renal vascular endothelial cells to maintain normal renal function. Additionally, ischemic injury to vascular smooth muscles cells and endothelial cells during the initiation phase can also contribute to the structural abnormalities observed in the renal vasculature during ischemic AKI.

The extension phase is characterized by continued hypoxia following the initial ischemic event and an inflammatory response. Both events are pronounced in the corticomedullary junction (CMJ), or outer medullary region, of the kidney. During this phase, renal vascular endothelial cell damage likely plays a key role in the continued ischemia of the renal tubular epithelium, as well as the inflammatory response observed in ischemic acute renal failure. During this phase, cells continue to undergo injury and death with both necrosis and apoptosis being present predominantly in the outer medulla. In contrast, the proximal tubule cells in the outer cortex, where blood flow has returned to near normal levels, undergo cellular repair and improve morphologically during this phase. As cellular injury continues in the CMJ region during the extension phase, the GFR continues to fall. Continued production and release of chemokines and cytokines further enhance the inflammatory cascade.

AKI from glomerular damage occurs in severe cases of acute glomerulonephritis (GN). Acute GN can be due to a primary renal disease, such as an idiopathic rapidly progressive GN or as part of a systemic disease, such as systemic lupus erythematosus, bacterial endocarditis, or Wegener's granulomatosis.

AKI from interstitial damage can result from acute interstitial nephritis due to an allergic reaction to medications (for example, penicillins, cephalosporins, and sulfonamides) or infection (for example, bacterial infections, such as leptospirosis, legionella, rarely pyelonephritis, and viral infections, such as Hanta virus).

AKI from vascular damage occurs because injury to intrarenal vessels decreases renal perfusion and diminishes GFR. Causes of vascular injury include malignant hypertension, atheroembolic disease, preeclampsia/eclampsia, and hemolyticuremic syndrome (HUS)/thrombotic thrombocytopenia purpura (TTP).

HPTPβ Inhibitors.

Compounds disclosed herein can be effective as HPTP-β inhibitors and Tie-2 activators. The compounds can promote that activity, for example, by binding to or inhibiting HPTPβ. Such compounds can bind to HPTPβ, for example, by mimicking the binding mechanism of a native substrate, such as a phosphorylated compound. A compound can be a phosphate mimetic or bioisostere, for example, a sulfamic acid. The compound could also be derived from an amino acid building block or comprise an amino acid backbone for efficiency and economy of synthesis.

In some embodiments, a compound disclosed herein is a compound of the formula:

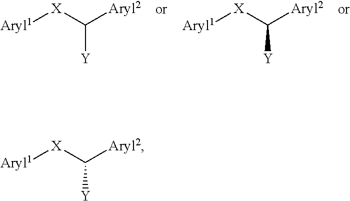

wherein:
Aryl¹ is an aryl group which is substituted or unsubstituted; Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

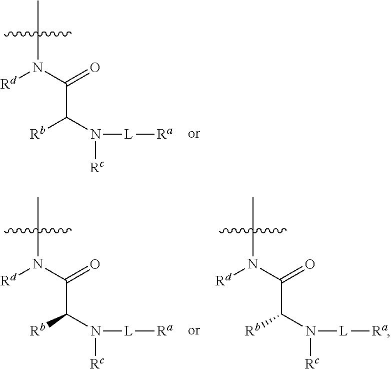

wherein:
L is alkylene, alkenylene, or alkynylene, any of which is substituted or unsubstituted, or together with the nitrogen atom to which L is bound forms an amide linkage, a carbamate linkage, or a sulfonamide linkage, or a chemical bond, or together with any of R^{a}, R^{b}, R^{c}, and R^{d} forms a ring that is substituted or unsubstituted; R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L, R^{b}, R^{c}, and R^{d} forms a ring that is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L, R^{a}, R^{c}, and R^{d} forms a ring that is substituted or unsubstituted; R^{c} is H or alkyl which is substituted or unsubstituted, or together with any of L, R^{a}, R^{b}, and R^{d} forms a ring that is substituted or unsubstituted; R^{d} is H or alkyl which is substituted or unsubstituted, or together with any of L, R^{a}, R^{b}, and R^{c} forms a ring that is substituted or unsubstituted; and R^{g} is H, alkyl, alkenyl, alkynyl, aryl, arylalkyl, heterocyclyl, heterocyclylalkyl, heteroaryl, or heteroarylalkyl, any of which is substituted or unsubstituted, or a pharmaceutically-acceptable salt, tautomer, or zwitterion thereof.

In some embodiments, aryl¹ is substituted or unsubstituted phenyl, aryl² is substituted or unsubstituted heteroaryl, and X is alkylene. In some embodiments, aryl¹ is substituted phenyl, aryl² is substituted heteroaryl, and X is methylene.

In some embodiments, a compound is of the formula:

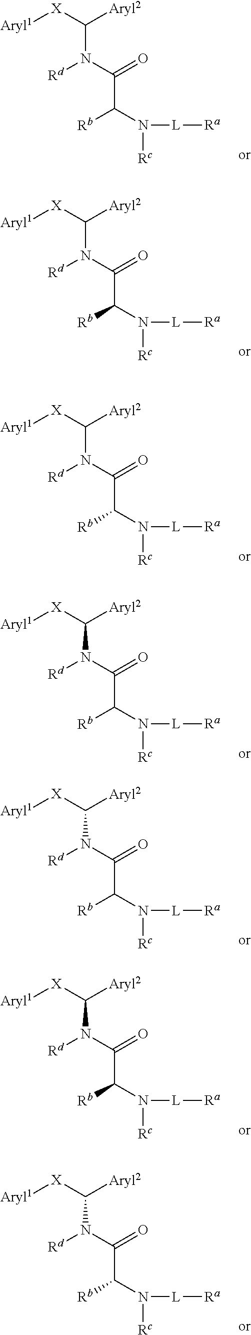

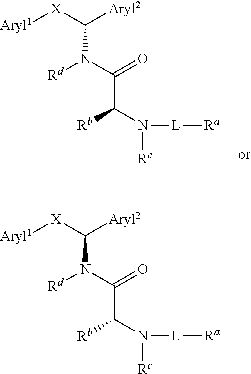

wherein aryl¹ is para-substituted phenyl, aryl² is substituted heteroaryl; X is methylene; L is alkylene, alkenylene, or alkynylene, any of which is substituted or unsubstituted, or together with the nitrogen atom to which L is bound forms an amide linkage, a carbamate linkage, or a sulfonamide linkage, or a chemical bond; R^{a} is H, alkyl, alkenyl, alkynyl, aryl, arylalkyl, heterocyclyl, heterocyclylalkyl, heteroaryl, or heteroarylalkyl, any of which is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted; R^{c} is H or alkyl which is substituted or unsubstituted; and R^{d} is H or alkyl which is substituted or unsubstituted.

In some embodiments, aryl¹ is para-substituted phenyl; aryl² is a substituted thiazole moiety; X is methylene; L together with the nitrogen atom to which L is bound forms a carbamate linkage; R^{a} is alkyl, which is substituted or unsubstituted; R^{b} is arylalkyl, which is substituted or unsubstituted; R^{e} is H; and R^{d} is H.

In some embodiments, Aryl² is:

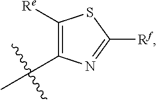

wherein R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted. In some embodiments, R^{e} is H, OH, F, Cl, Br, I, alkyl, or an alkoxy group, any of which is substituted or unsubstituted and R^{f} is alkyl, aryl, heterocyclyl, or heteroaryl, any of which is substituted or unsubstituted. In some embodiments, aryl¹ is 4-phenylsulfamic acid; R^{a} is alkyl, which is substituted or unsubstituted; R^{b} is arylalkyl, which is substituted or unsubstituted; R^{e} is H; and R^{f} is heteroaryl. In some embodiments, aryl¹ is 4-phenylsulfamic acid; R^{a} is alkyl; which is substituted or unsubstituted; R^{b} is arylalkyl, which is substituted or unsubstituted; R^{e} is H; and R^{f} is alkyl.

In some embodiments, Aryl² is:

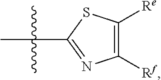

wherein R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted, R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted. In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted. In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted. In some embodiments, aryl¹ is 4-phenylsulfamic acid; R^{a} is alkyl, which is substituted or unsubstituted; R^{b} is arylalkyl, which is substituted or unsubstituted; R^{e} is H; and R^{f} is heteroaryl.

In some embodiments, a substituted phenyl group is:

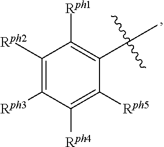

wherein:
each of R^{ph1}, R^{ph2}, R^{ph3}, R^{ph4}, and R^{ph5} is independently H, OH, F, Cl, Br, I, CN, sulfamic acid, tosylate, mesylate, triflate, besylate, alkyl, alkenyl, alkynyl, an alkoxy group, a sulfhydryl group, a nitro group, an azido group, a sulfoxide group, a sulfone group, a sulfonamide group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl.

Illustrative compounds include the following:

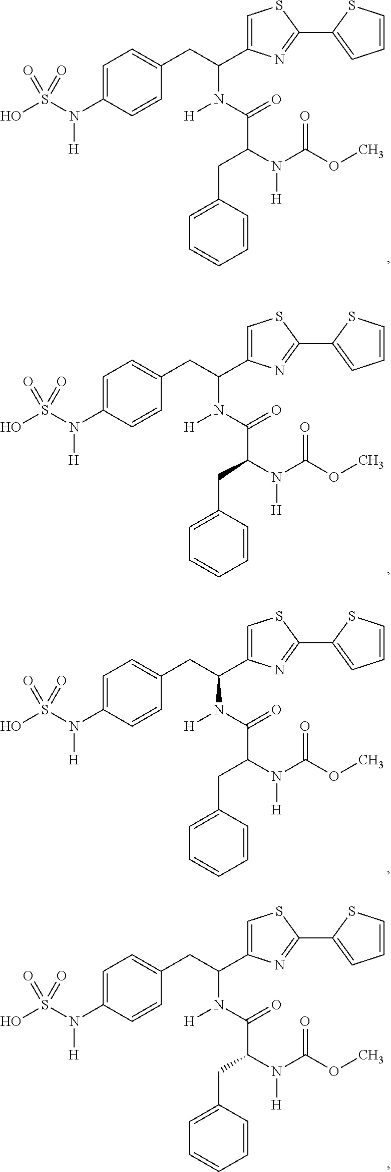 [SMILES: COC(=O)NC(Cc1ccccc1)C(=O)NC(Cc1ccc(NS(=O)(=O)O)cc1)c1csc(-c2cccs2)n1 ; COC(=O)N[C@@H](Cc1ccccc1)C(=O)NC(Cc1ccc(NS(=O)(=O)O)cc1)c1csc(-c2cccs2)n1 ; COC(=O)NC(Cc1ccccc1)C(=O)N[C@@H](Cc1ccc(NS(=O)(=O)O)cc1)c1csc(-c2cccs2)n1 ; COC(=O)N[C@H](Cc1ccccc1)C(=O)NC(Cc1ccc(NS(=O)(=O)O)cc1)c1csc(-c2cccs2)n1]

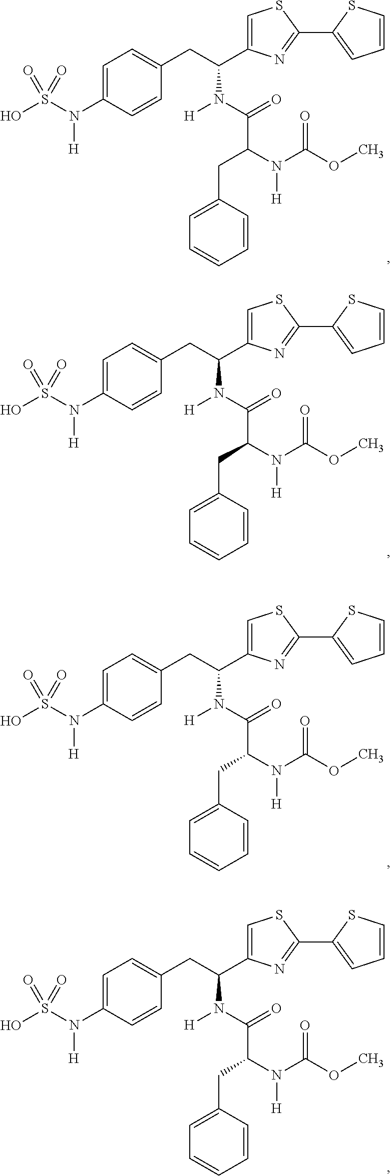 [SMILES: COC(=O)NC(Cc1ccccc1)C(=O)N[C@H](Cc1ccc(NS(=O)(=O)O)cc1)c1csc(-c2cccs2)n1 ; COC(=O)N[C@@H](Cc1ccccc1)C(=O)N[C@@H](Cc1ccc(NS(=O)(=O)O)cc1)c1csc(-c2cccs2)n1 ; COC(=O)N[C@H](Cc1ccccc1)C(=O)N[C@H](Cc1ccc(NS(=O)(=O)O)cc1)c1csc(-c2cccs2)n1 ; COC(=O)N[C@H](Cc1ccccc1)C(=O)N[C@@H](Cc1ccc(NS(=O)(=O)O)cc1)c1csc(-c2cccs2)n1]

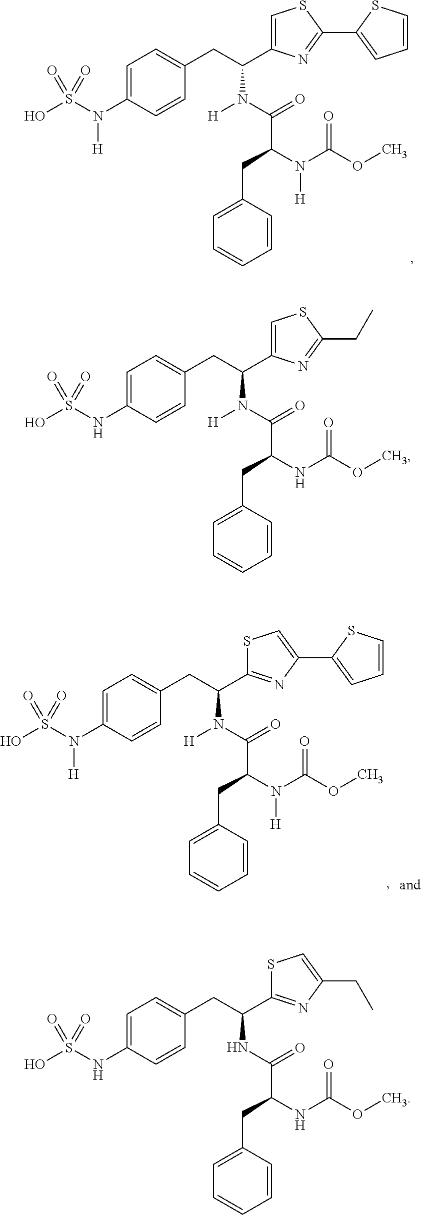 [SMILES: COC(=O)N[C@@H](Cc1ccccc1)C(=O)N[C@H](Cc1ccc(NS(=O)(=O)O)cc1)c1csc(-c2cccs2)n1 ; CCc1nc([C@H](Cc2ccc(NS(=O)(=O)O)cc2)NC(=O)[C@H](Cc2ccccc2)NC(=O)OC)cs1 ; COC(=O)N[C@@H](Cc1ccccc1)C(=O)N[C@@H](Cc1ccc(NS(=O)(=O)O)cc1)c1nc(-c2cccs2)cs1 ; CCc1csc([C@H](Cc2ccc(NS(=O)(=O)O)cc2)NC(=O)[C@H](Cc2ccccc2)NC(=O)OC)n1]

### Optional Substituents for Chemical Groups.

Non-limiting examples of optional substituents include hydroxyl groups, sulfhydryl groups, halogens, amino groups, nitro groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, and ester groups.

Non-limiting examples of alkyl and alkylene groups include straight, branched, and cyclic alkyl and alkylene groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl.

An aryloxy group can be, for example, an oxygen atom substituted with any aryl group, such as phenoxy.

An aralkyl group can be, for example, any alkyl group substituted with any aryl group, such as benzyl.

An arylalkoxy group can be, for example, an oxygen atom substituted with any aralkyl group, such as benzyloxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

An acyl group can be, for example, a carbonyl group substituted with hydrocarbyl, alkyl, hydrocarbyloxy, alkoxy, aryl, aryloxy, aralkyl, arylalkoxy, or a heterocycle. Non-limiting examples of acyl include acetyl, benzoyl, benzyloxycarbonyl, phenoxycarbonyl, methoxycarbonyl, and ethoxycarbonyl.

An acyloxy group can be an oxygen atom substituted with an acyl group. An ester or an ester group comprises an acyloxy group. A non-limiting example of an acyloxy group, or an ester group, is acetate.

A carbamate group can be an oxygen atom substituted with a carbamoyl group, wherein the nitrogen atom of the carbamoyl group is unsubstituted, monosubstituted, or disubstituted with one or more of hydrocarbyl, alkyl, aryl, heterocyclyl, or aralkyl. When the nitrogen atom is disubstituted, the two substituents together with the nitrogen atom can form a heterocycle.

### Pharmaceutically-Acceptable Salts.

The present disclosure provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the present disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, piprazole, imidazole, or pyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a piprazole salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

A compound herein can be a salt of an acidic group, for example:

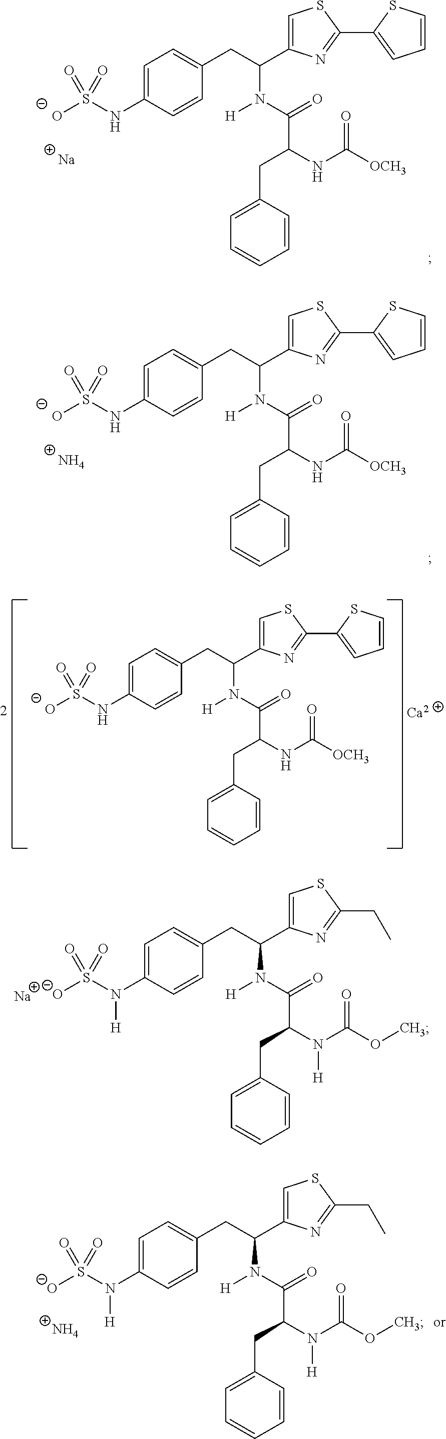

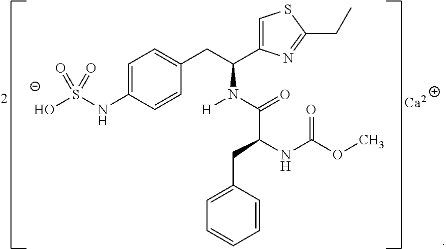

A compound herein can be a salt of a basic group formed from a strong acid, for example:

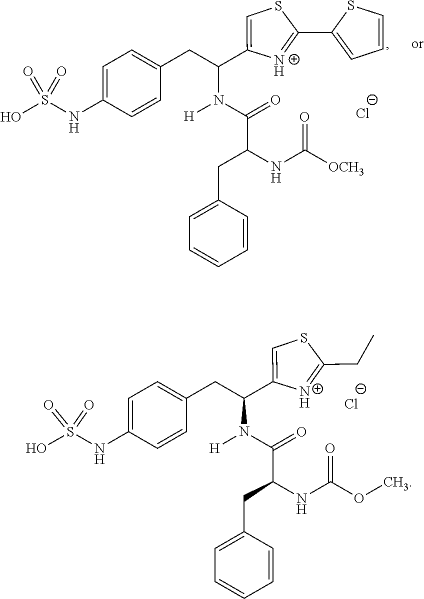 [SMILES: COC(=O)NC(Cc1ccccc1)C(=O)NC(Cc1ccc(NS(=O)(=O)O)cc1)c1csc(-c2cccs2)[nH+]1 ; [Cl-] ; CCc1[nH+]c([C@H](Cc2ccc(NS(=O)(=O)O)cc2)NC(=O)[C@H](Cc2ccccc2)NC(=O)OC)cs1 ; [Cl-]]

A compound herein can also exist in a zwitterionic form, for example:

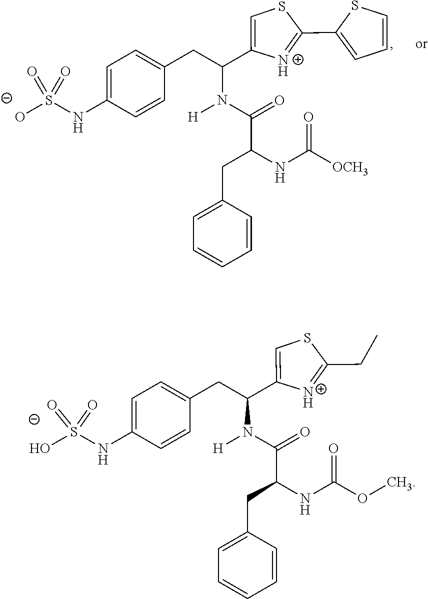

### Formulations.

A pharmaceutical composition of the disclosure can provide a therapeutically-effective amount of an inhibitor of HPTPβ. A pharmaceutical composition of the disclosure can provide a therapeutically-effective amount of an activator of Tie-2.

The disclosed formulations can comprise one or more pharmaceutically-acceptable agents, which alone or in combination solubilize a compound herein or a pharmaceutically-acceptable salt thereof.

In some embodiments, a compound or pharmaceutically-acceptable salt thereof is present in a formulation in an amount of from about 0.1 mg/mL to about 100 mg/mL, from about 0.1 mg/mL to about 1 mg/mL, from about 0.1 mg/mL to about 5 mg/mL, from about 5 mg/mL to about 10 mg/mL, from about 10 mg/mL to about 15 mg/mL, from about 15 mg/mL to about 20 mg/mL, from about 20 mg/mL to about 25 mg/mL, from about 25 mg/mL to about 30 mg/mL, from about 30 mg/mL to about 35 mg/mL, from about 35 mg/mL to about 40 mg/mL, from about 40 mg/mL to about 45 mg/mL, about 45 mg/mL to about 50 mg/mL, from about 50 mg/mL to about 55 mg/mL, from about 55 mg/mL to about 60 mg/mL, from about 60 mg/mL to about 65 mg/mL, from about 65 mg/mL to about 70 mg/mL, from about 70 mg/mL to about 75 mg/mL, about 75 mg/mL to about 80 mg/mL, from about 80 mg/mL to about 85 mg/mL, from about 85 mg/mL to about 90 mg/mL, from about 90 mg/mL to about 95 mg/mL, or from about 95 mg/mL to about 100 mg/mL.

In some embodiments, a compound or pharmaceutically-acceptable salt thereof is present in a formulation in an amount of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL about 52 mg/mL, about 53 mg/mL, about 54 mg/mL, about 55 mg/mL, about 56 mg/mL, about 57 mg/mL, about 58 mg/mL, about 59 mg/mL, about 60 mg/mL, about 61 mg/mL about 62 mg/mL, about 63 mg/mL, about 64 mg/mL, about 65 mg/mL, about 66 mg/mL, about 67 mg/mL, about 68 mg/mL, about 69 mg/mL, about 70 mg/mL, about 71 mg/mL about 72 mg/mL, about 73 mg/mL, about 74 mg/mL, about 75 mg/mL, about 76 mg/mL, about 77 mg/mL, about 78 mg/mL, about 79 mg/mL, about 80 mg/mL, about 81 mg/mL about 82 mg/mL, about 83 mg/mL, about 84 mg/mL, about 85 mg/mL, about 86 mg/mL, about 87 mg/mL, about 88 mg/mL, about 89 mg/mL, about 90 mg/mL, about 91 mg/mL about 92 mg/mL, about 93 mg/mL, about 94 mg/mL, about 95 mg/mL, about 96 mg/mL, about 97 mg/mL, about 98 mg/mL, about 99 mg/mL, or about 100 mg/mL.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

A formulation that is disclosed herein can be made more soluble by the addition of an additive or agent, for example, a cyclodextrin moiety. In some embodiments, the agent that improves aqueous solubility of a compound of the disclosure is a 2-hydroxypropyl-β-cyclodextrin moiety or a sulfobutylether-β-cyclodextrin moiety. The improvement of solubility of the formulation can increase by about 5%, about 10%, about 15%, about 20%, about 25%, about 30%, about 35%, about 40%, about 45%, about 50%, about 55%, about 60%, about 65%, about 70%, about 75% about 80%, about 85%, about 90%, about 95%, about 100%, about 110%, about 120%, about 130%, about 140%, about 150%, about 160%, about 170%, about 180%, about 190%, about 200%, about 225%, about 250%, about 275%, about 300%, about 325%, about 350%, about 375%, about 400%, about 450%, or about 500%.

A formulation disclosed herein can be stable for about 1 day, about 2 days, about 3 days, about 4 days, about 5 days, about 6 days, about 7 days, about 8 days, about 9 days, about 10 days, about 2 weeks, about 4 weeks, about 6 weeks, about 8 weeks, about 10 weeks, about 12 weeks, about 3 months, about 4 months, about 5 months, about 6 months, about 7 months, about 8 months, about 9 months, about 10 months, about 11 months, or about one year. A formulation disclosed herein can be stable, for example, at about 0° C., about 5° C., about 10° C., about 15° C., about 20° C., about 25° C., about 30° C., about 35° C., about 40° C., about 45° C., about 50° C., about 60° C., about 70° C., or about 80° C.

a. Alcohols.

A non-limiting example of a solubilizing agent includes an organic solvent. Non-limiting examples of organic solvents include alcohols, for example, C₁-C₄ linear alkyl, C₃-C₄ branched alkyl, ethanol, ethylene glycol, glycerin, 2-hydroxypropanol, propylene glycol, maltitol, sorbitol, xylitol; substituted or unsubstituted aryl, and benzyl alcohol.

b. Cyclodextrins.

Non-limiting examples of cyclodextrins include β-cyclodextrin, methyl β-cyclodextrin, 2-hydroxypropyl-β-cyclodextrin (HPβCD), sulfobutyl ether-β-cyclodextrin sodium salt, hydroxyethyl-β-cyclodextrin (HE-β-CD), heptakis (2,6-di-O-methyl)-O-cyclodextrin (DMβCD), 2-hydroxypropyl-β-cyclodextrin, α-cyclodextrin, γ-cyclodextrin, 2-hydroxypropyl-γ-cyclodextrin (HPγCD), and sulfobutylether-β-cyclodextrin (SBECD) sodium salt. A cyclodextrin can possess a large cyclic structure with a channel passing through the center of the structure. The interior of the cyclodextrin can be hydrophobic, and interact favorably with hydrophobic molecules. The exterior of the cyclodextrin can be highly hydrophilic owing to the several hydroxyl groups exposed to bulk solvent. Capture of a hydrophobic molecule, such as a compound disclosed herein, in the channel of the cyclodextrin can result in the formation of a complex stabilized by non-covalent hydrophobic interactions. The complex can be soluble in water, and carry the captured hydrophobic molecule into the bulk solvent.

Formulations of the disclosure can comprise randomly methylated β-cyclodextrins (RAMEB or RMCD). The formulations of the disclosure can comprise RAMEB comprising at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21 methyl groups.

The disclosed solubilizing systems comprise 2-hydroxypropyl-beta-cyclodextrin (HPβCD). 2-Hydroxypropyl-β-cyclodextrin [CAS No. 128446-35-5] is commercially available as Cavitron™. 2-Hydroxypropyl-3-cyclodextrin, also described known as hydroxypropyl-3-cyclodextrin, 2-hydroxypropyl-beta-cyclodextrin, hydroxypropyl-beta-cyclodextrin or HPβCD, can be represented by either of the following formulae:

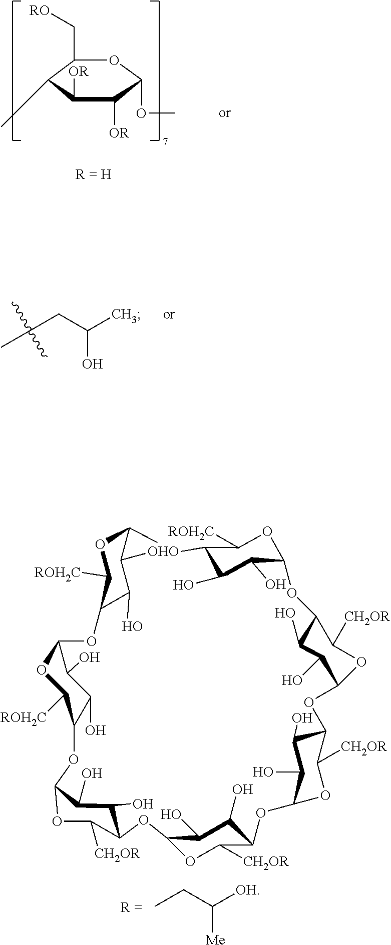

The average molecular weight of Cavitron™, is approximately 1396 Da, wherein the average degree of substitution is from about 0.5 to about 1.3 units of 2-hydroxypropyl per ring glucose unit.

In one embodiment, a formulation disclosed herein can comprise a ratio of about 20 parts of a compound herein or a pharmaceutically-acceptable salt thereof to about 1 part solubilizing system (about 20:about 1), to about 1 part of the compound herein or a pharmaceutically-acceptable salt thereof to about 20 parts solubilizing system (about 1:about 20). For example, a formulation containing about 100 mg of a compound herein or a pharmaceutically-acceptable salt thereof can contain from about 5 mg to about 2000 mg of a solubilizing agent, such as a cyclodextrin. In another embodiment, the ratio can be based on number, or moles, or compound compared to number, or moles, of the solubilizing system.

The following are non-limiting examples of ratios of a compound herein and a solubilizing agent, such as a cyclodextrin. The following examples alternatively describe the ratio of a solubilizing agent, such as a cyclodextrin, and a compound herein. The ratio can be: about 20:about 1; about 19.9:about 1; about 19.8:about 1; about 19.7:about 1; about 19.6: about 1; about 19.5:about 1; about 19.4:about 1; about 19.3:about 1; about 19.2:about 1; about 19.1:about 1; about 19:about 1; about 18.9:about 1; about 18.8:about 1; about 18.7:about 1; about 18.6:about 1; about 18.5:about 1; about 18.4:about 1; about 18.3:about 1; about 18.2:about 1; about 18.1:about 1; about 18:about 1; about 17.9:about 1; about 17.8:about 1; about 17.7:about 1; about 17.6:about 1; about 17.5:about 1; about 17.4:about 1; about 17.3:about 1; about 17.2:about 1; about 17.1:about 1; about 17:about 1; about 16.9 about 1; about 16.8:about 1; about 16.7:about 1; about 16.6:about 1; about 16.5:about 1; about 16.4:about 1; about 16.3:about 1; about 16.2:about 1; about 16.1:about 1; about 16:about 1; about 15.9:about 1; about 15.8:about 1; about 15.7:about 1; about 15.6:about 1; about 15.5:about 1; about 15.4:about 1; about 15.3:about 1; about 15.2:about 1; about 15.1:about 1; about 15:about 1; about 14.9:about 1; about 14.8:about 1; about 14.7:about 1; about 14.6:about 1; about 14.5:about 1; about 14.4:about 1; about 14.3:about 1; about 14.2:about 1; about 14.1:about 1; about 14:about 1; about 13.9:about 1; about 13.8:about 1; about 13.7:about 1; about 13.6:about 1; about 13.5:about 1; about 13.4:about 1; about 13.3:about 1; about 13.2:about 1; about 13.1:about 1; about 13:about 1; about 12.9:about 1; about 12.8:about 1; about 12.7:about 1; about 12.6:about 1; about 12.5:about 1; about 12.4:about 1; about 12.3:about 1; about 12.2:about 1; about 12.1:about 1; about 12:about 1; about 11.9:about 1; about 11.8:about 1; about 11.7:about 1; about 11.6:about 1; about 11.5:about 1; about 11.4:about 1; about 11.3:about 1; about 11.2:about 1; about 11.1:about 1; about 11:about 1; about 10.9:about 1; about 10.8:about 1; about 10.7:about 1; about 10.6:about 1; about 10.5:about 1; about 10.4:about 1; about 10.3:about 1; about 10.2:about 1; about 10.1:about 1; about 10:about 1; about 9.9:about 1; about 9.8:about 1; about 9.7:about 1; about 9.6:about 1; about 9.5:about 1; about 9.4:about 1; about 9.3:about 1; about 9.2:about 1; about 9.1:about 1; about 9:about 1; about 8.9:about 1; about 8.8:about 1; about 8.7:about 1; about 8.6:about 1; about 8.5:about 1; about 8.4:about 1; about 8.3:about 1; about 8.2:about 1; about 8.1:about 1; about 8:about 1; about 7.9:about 1; about 7.8:about 1; about 7.7:about 1; about 7.6:about 1; about 7.5:about 1; about 7.4:about 1; about 7.3:about 1; about 7.2:about 1; about 7.1:about 1; about 7:about 1; about 6.9:about 1; about 6.8:about 1; about 6.7:about 1; about 6.6:about 1; about 6.5:about 1; about 6.4:about 1; about 6.3:about 1; about 6.2 about 1; about 6.1:about 1; about 6:about 1; about 5.9:about 1; about 5.8:about 1; about 5.7:about 1; about 5.6:about 1; about 5.5:about 1; about 5.4:about 1; about 5.3:about 1; about 5.2:about 1; about 5.1:about 1; about 5:about 1; about 4.9:about 1; about 4.8:about 1; about 4.7:about 1; about 4.6:about 1; about 4.5:about 1; about 4.4:about 1; about 4.3:about 1; about 4.2:about 1; about 4.1:about 1; about 4:about 1; about 3.9:about 1; about 3.8:about 1; about 3.7:about 1; about 3.6:about 1; about 3.5:about 1; about 3.4:about 1; about 3.3:about 1; about 3.2:about 1; about 3.1:about 1; about 3:about 1; about 2.9:about 1; about 2.8:about 1; about 2.7:about 1; about 2.6:about 1; about 2.5:about 1; about 2.4:about 1; about 2.3:about 1; about 2.2:about 1; about 2.1:about 1; about 2:about 1; about 1.9:about 1; about 1.8:about 1; about 1.7:about 1; about 1.6:about 1; about 1.5:about 1; about 1.4:about 1; about 1.3:about 1; about 1.2:about 1; about 1.1:about 1; or about 1:about 1.

c. Polyvinylpyrrolidione.

Another non-limiting example of a solubilizing agent is polyvinylpyrrolidone (PVP), having the formula:

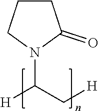

wherein the index n is from about 40 to about 200. PVP's can have an average molecular weight from about 5500 to about 28,000 g/mol. One non-limiting example is PVP-10, having an average molecular weight of approximately 10,000 g/mol.
d. Polyakyleneoxides and Ethers Thereof.

Another non-limiting example of solubilizing agents includes polyalkyleneoxides, and polymers of alcohols or polyols. Polymers can be mixed, or contain a single monomeric repeat subunit. For example, polyethylene glycols having an average molecular weight of from about 200 to about 20,000, for example, PEG 200, PEG 400, PEG 600, PEG 1000, PEG 1450, PEG 1500, PEG 4000, PEG 4600, and PEG 8000. In a same embodiment, a composition comprises one or more polyethylene glycols chosen from PEG 400, PEG 1000, PEG 1450, PEG 4600 and PEG 8000.

Other polyalkyleneoxides are polypropylene glycols having the formula:

HO[CH(CH₃)CH₂O]ₓH

wherein the index x represents the average number of propyleneoxy units in the polymer. The index x can be represented by a whole number or a fraction. For example, a polypropylene glycol having an average molecular weight of 8,000 g/mole (PEG 8000) can be represented by the formulae:

HO[CH(CH₃)CH₂O]₁₃₈H or HO[CH(CH₃)CH₂O]_{137.6}H

or the polypropylene glycol can be represented by the common, short hand notation: PEG 8000.

Another example of polypropylene glycols can have an average molecular weight from about 1200 g/mol to about 20,000 g/mol, i.e., a polypropylene glycol having an average molecular weight of about 8,000 g/mol, for example, PEG 8000.

Another solubilizing agent is Polysorbate 80 (Tween™ 80), which is an oleate ester of sorbitol and its anhydrides copolymerized with approximately 20 moles of ethylene oxide for each mole of sorbitol and sorbitol anhydrides. Polysorbate 80 is made up of sorbitan mono-9-octadecanoate poly(oxy-1,2-ethandiyl) derivatives.

Solubilizing agents also include poloxamers having the formula:

HO(CH₂CH₂)_{y1}(CH₂CH₂CH₂O)_{y2}(CH₂CH₂O)_{y3}OH

which are nonionic block copolymers composed of a polypropyleneoxy unit flanked by two polyethyleneoxy units. The indices y¹, y², and y³ have values such that the poloxamer has an average molecular weight of from about 1000 g/mol to about 20,000 g/mol.
f. Excipients.

A pharmaceutical composition of the present disclosure can be a combination of any pharmaceutical compounds described herein with other chemical components, such as carriers, stabilizers, diluents, dispersing agents, suspending agents, thickening agents, or excipients. The pharmaceutical composition facilitates administration of the compound to an organism. Pharmaceutical compositions can be administered in therapeutically-effective amounts as pharmaceutical compositions by various forms and routes including, for example, intravenous, intravitreal, subcutaneous, intramuscular, oral, rectal, aerosol, parenteral, ophthalmic, pulmonary, transdermal, vaginal, optic, nasal, and topical administration.

A pharmaceutical composition can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated readily by combining the active compounds with pharmaceutically-acceptable carriers or excipients. Such carriers can be used to formulate tablets, powders, pills, dragees, capsules, liquids, gels, syrups, elixirs, slurries, suspensions and the like, for oral ingestion by a subject.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used, which can contain an excipient such as gum 28yrazi, talc, polyvinylpyrrolidone, carbopol gel, polyethylene glycol, or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline-processed. The push-fit capsules can contain the active ingredients in admixture with filler such as lactose, binders such as starches, or lubricants such as talc or magnesium stearate and, stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration.

For buccal or sublingual administration, the compositions can be tablets, lozenges, or gels.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

Formulations suitable for transdermal administration of the active compounds can employ transdermal delivery devices and transdermal delivery patches, and can be lipophilic emulsions or buffered aqueous solutions, dissolved or dispersed in a polymer or an adhesive. Such patches can be constructed for continuous, pulsatile, or on demand delivery of pharmaceutical compounds. Transdermal delivery can be accomplished by means of iontophoretic patches. Additionally, transdermal patches can provide controlled delivery. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically-acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and adhesives to secure the device to the skin or the eye.

For administration by inhalation, the active compounds can be in a form as an aerosol, a mist, or a powder. Pharmaceutical compositions are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compounds and a suitable powder base such as lactose or starch.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone and PEG. In suppository forms of the compositions, a low-melting wax such as a mixture of fatty acid glycerides or cocoa butter can be used.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compounds described herein can be manufactured, for example, by mixing, dissolving, granulating, dragee-making, levigating, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. The methods and pharmaceutical compositions described herein include the use of crystalline forms (also known as polymorphs), and active metabolites of these compounds having the same type of activity.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, cachets, and suppositories. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the present disclosure include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

The individual dose administered to a subject can be about 0.5 mg, about 1 mg, about 2 mg, about 3 mg, about 4 mg, about 5 mg, about 6 mg, about 7 mg, about 8 mg, about 9 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, or about 500 mg of a compound of the present disclosure. The individual dose administered to a subject can be from about 0.1 mg to about 25 mg, about 0.1 mg to about 50 mg, about 0.1 mg to about 75 mg, or about 0.1 mg to about 100 mg. The individual dose administered to a subject can be from about 0.5 mg to about 10 mg, about 0.5 mg to about 20 mg, or about 0.5 mg to about 30 mg. In some embodiments, the individual dose administered to a subject can be about 10 mg of a compound of the present disclosure. In some embodiments, the individual dose administered to a subject can be about 15 mg of a compound of the present disclosure. In some embodiments, the individual dose administered to a subject can be about 20 mg of a compound of the present disclosure. In some embodiments, the individual dose administered to a subject can be about 30 mg of a compound of the present disclosure. In some embodiments, the individual dose of a compound of the present disclosure administered to a subject can be about 15 mg twice per day or about 30 mg per day.

Non-limiting examples of pharmaceutically-acceptable excipients suitable for use in the present disclosure include granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents, anti-microbial agents, plant cellulosic material and spheronization agents, and any combination thereof.

A composition of the present disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that drug release rates and drug release profiles can be matched to physiological and chronotherapeutic requirements or, alternatively, has been formulated to effect release of a drug at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

The disclosed compositions can optionally comprise from about 0.001% to about 0.005% weight by volume pharmaceutically-acceptable preservatives. One non-limiting example of a suitable preservative is benzyl alcohol.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

The disclosed methods include administration of an HPTPβ inhibitor, or a pharmaceutically-acceptable salt thereof, in combination with a pharmaceutically-acceptable carrier. The carrier can be selected to minimize any degradation of the active ingredient and to minimize any adverse side effects in the subject.

The disclosed methods include administration of a Tie-2 activator, or a pharmaceutically-acceptable salt thereof, in combination with a pharmaceutically-acceptable carrier. The carrier can be selected to minimize any degradation of the active ingredient and to minimize any adverse side effects in the subject.

The Tie-2 activator or a pharmaceutically-acceptable salt thereof herein can be conveniently formulated into pharmaceutical compositions composed of one or more pharmaceutically-acceptable carriers. See e.g., Remington's Pharmaceutical Sciences, latest edition, by E.W. Martin Mack Pub. Co., Easton, Pa., which discloses typical carriers and conventional methods of preparing pharmaceutical compositions that can be used in conjunction with the preparation of formulations of the compound described herein and which is incorporated by reference herein. Such pharmaceuticals can be standard carriers for administration of compositions to humans and non-humans, including solutions such as sterile water, saline, and buffered solutions at physiological pH. Other compositions can be administered according to standard procedures. For example, pharmaceutical compositions can also include one or more additional active ingredients such as antimicrobial agents, anti-inflammatory agents, and anesthetics.

Non-limiting examples of pharmaceutically-acceptable carriers include saline solution, Ringer's solution and dextrose solution. The pH of the solution can be from about 5 to about 8, and can be from about 7 to about 7.5. Further carriers include sustained release preparations such as semipermeable matrices of solid hydrophobic polymers containing the Tie-2 activator or a pharmaceutically-acceptable salt thereof, where the matrices are in the form of shaped articles, such as films, liposomes, microparticles, and microcapsules.

The disclosed methods relate to administering the Tie-2 activator or a pharmaceutically-acceptable salt thereof as part of a pharmaceutical composition. The disclosed methods relate to administering the HPTPβ inhibitor or a pharmaceutically-acceptable salt thereof as part of a pharmaceutical composition. In various embodiments, compositions of the present disclosure can comprise a liquid comprising an active agent in solution, in suspension, or both. Liquid compositions can include gels. In one embodiment, the liquid composition is aqueous. Alternatively, the composition can take form of an ointment. In another embodiment, the composition is an in situ gellable aqueous composition. In some embodiments, the composition is an in situ gellable aqueous solution.

Pharmaceutical formulations can include additional carriers, as well as thickeners, diluents, buffers, preservatives, and surface active agents in addition to the compounds disclosed herein. Pharmaceutical formulations can also include one or more additional active ingredients such as antimicrobial agents, anti-inflammatory agents, anesthetics, and the like.

An excipient can fill a role as simple and direct as being an inert filler, or an excipient as used herein can be part of a pH stabilizing system or coating to insure delivery of the ingredients safely to the stomach.

The HPTPβ inhibitor or a pharmaceutically-acceptable salt thereof can also be present in liquids, emulsions, or suspensions for delivery of active therapeutic agents in aerosol form to cavities of the body such as the nose, throat, or bronchial passages. The ratio of HPTPβ inhibitor or a pharmaceutically-acceptable salt thereof to the other compounding agents in these preparations can vary as the dosage form requires.

Depending on the intended mode of administration, the pharmaceutical compositions administered as part of the disclosed methods can be in the form of solid, semi-solid or liquid dosage forms, such as, for example, tablets, suppositories, pills, capsules, powders, liquids, suspensions, lotions, creams, gels, for example, in unit dosage form suitable for single administration of a precise dosage. The compositions can contain, as noted above, an effective amount of the HPTPβ inhibitor or a pharmaceutically-acceptable salt thereof in combination with a pharmaceutically-acceptable carrier and, in addition, can include other medicinal agents, pharmaceutical agents, carriers, adjuvants, diluents, etc.

For solid compositions, nontoxic solid carriers include, for example, pharmaceutical grades of mannitol, lactose, starch, magnesium stearate, sodium saccharin, talc, cellulose, glucose, sucrose, and magnesium carbonate. In one embodiment, a composition comprising the HPTPβ inhibitor or a pharmaceutically-acceptable salt thereof in an amount of approximately 4 mg per 0.1 mL liquid is prepared. The liquid phase comprises sterile water and an appropriate amount of a saccharide or polysaccharide.

### Pharmaceutical Compositions.

Pharmaceutical compositions containing the compounds described herein can be administered for prophylactic or therapeutic treatments. Compositions can contain any number of active agents. In therapeutic applications, the compositions can be administered to a subject already suffering from a disease or condition, in an amount sufficient to cure or at least partially arrest the symptoms of the disease or condition, or to cure, heal, improve, reduce, lessen or ameliorate the disease or condition. Compounds can also be administered to lessen or reduce a likelihood of developing, contracting, or worsening a condition. Amounts effective for this use can vary based on the severity and course of the disease or condition, previous therapy, the subject's health status, weight, response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills or injections. The compounds can be packed together or separately, in a single package or in a plurality of packages. One or all of the therapeutic agents can be given in multiple doses. If not simultaneous, the timing between the multiple doses can vary.

Compounds and compositions described herein can be packaged as a kit. In some embodiments, the present disclosure provides a kit comprising a compound disclosed herein, or a pharmaceutically-acceptable salt thereof, and written instructions on use of the kit in the treatment of a condition described herein. In some embodiments, the present disclosure provides a kit comprising a compound disclosed herein, or a pharmaceutically-acceptable salt thereof, an antibody, and written instructions on use of the kit in the treatment of a condition described herein.

The compounds described herein can be administered before, during, or after the occurrence of a disease or condition, and the timing of administering the composition containing a compound can vary. For example, the compounds can be used as a prophylactic and can be administered continuously to subjects with a propensity to conditions or diseases in order to lessen or reduce a likelihood of the occurrence of the disease or condition. The compounds and compositions can be administered to a subject during or as soon as possible after the onset of the symptoms. The administration of the compounds can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for parenteral injection can be presented in unit dosage form, for example, in ampoules, or in multi-dose containers with a preservative.

An HPTPβ inhibitor described herein can be present in a composition in a range of from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, or from about 250 mg to about 300 mg.

An HPTPβ inhibitor described herein can be present in a composition in an amount of about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, or about 300 mg.

Treatment of Subjects with an HPTPβ Inhibitor.

The present disclosure discloses methods for treating a subject afflicted with diabetic nephropathy with an activator of Tie-2 or an inhibitor of HPTPβ. The subject can be a human. Treatment can include treating a human in a clinical trial. A treatment can comprise administering to a subject a pharmaceutical composition comprising one or more of the activators of Tie-2 described throughout the disclosure. A treatment can comprise administrating to a subject a therapy that promotes the phosphorylation of a Tie-2 molecule.

The present disclosure discloses methods for treating a subject afflicted with acute kidney injury with a therapeutically-effective amount of an activator of Tie-2 or an inhibitor of HPTPβ. The subject can be a human. Treatment can include treating a human in a clinical trial. A treatment can comprise administering to a subject a pharmaceutical composition comprising one or more of the activators of Tie-2 described throughout the disclosure. A treatment can comprise administrating to a subject a therapy that promotes the phosphorylation of a Tie-2 molecule. A therapeutically-effective amount can be from about 0.1 mg to about 100 mg or from about 0.5 mg to about 30 mg.

Non-limiting examples of possible subjects for administration include the following. Subjects can be humans, non-human primates such as chimpanzees, and other apes and monkey species; farm animals such as cattle, horses, sheep, goats, and swine; domestic animals such as rabbits, dogs, and cats; and laboratory animals including rats, mice, and guinea pigs. A subject can be of any age. Subjects can be, for example, elderly adults, adults, adolescents, pre-adolescents, children, toddlers, and infants.

Some conditions can lead to an increase in the levels of Ang-2, altering the ratio of Ang-1/Ang-2 in circulation. In some aspects, a therapy can improve the outcome of a disease state by altering the ratio of Ang-1/Ang-2 in circulation. A therapy can provide an Ang-1/Ang-2 ratio or an Ang-2/Ang-1 ratio of about 1:about 1, about 2:about 1, about 3:about 1, about 4:about 1, about 5:about 1, about 6:about 1, about 7:about 1, about 8:about 1, about 9:about 1, or about 10:about 1.

### Experimental Methods.

Methods of measuring kidney function include blood and urine tests, which can demonstrate the performance of a subject's kidneys, the presence and/or extent of kidney disease, and the progression of kidney disease over time. Tests to measure kidney function include, for example, the blood urea nitrogen (BUN) test.

The liver produces ammonia, which contains nitrogen, after the liver breaks down proteins used by the body's cells. The nitrogen from the ammonia combines with other elements in the body to form urea, which is a chemical waste product. The urea travels from the subject's kidneys through the bloodstream. Healthy kidneys filter urea and remove other waste products from the blood, and filtered waste products leave the body through urine.

The BUN test measures the amount of urea nitrogen in a subject's blood, revealing information about the subject's kidney and liver function. A high BUN test result can indicate the presence of kidney disease, kidney failure, dehydration, obstruction in the urinary tract, heart disease, cognitive heart failure, gastrointestinal bleeding, high protein levels, stress, or shock. Lower BUN levels can indicate liver failure, malnutrition, severe lack of protein in the diet, or overhydration. In some embodiments, a colorimetric BUN assay can be used to quantify urea nitrogen in serum, plasma, urine, saliva, or tissue culture media samples. Colorimetric BUN assays use a urea nitrogen standard and color reagents to quantify the amount of urea nitrogen in a biological sample.

Additional tests used to test kidney function include, for example, urinalysis, a serum creatinine (sCr) test, and measurements of estimated glomerular filtration rates (eGFR) or albumin-to-creatinine ratios (ACR). The sCr test measures the amount of creatinine, a waste product produced from the normal wear and tear on muscles, in a subject's blood. eGFR is calculated using serum creatinine and other factors, such as age, race, and gender, and are used to account for possible differences in creatinine levels between people that measuring sCr alone does not. ACR is used to measure the amount of albumin in a subject's urine, and is calculated by dividing the amount of urine albumin by the amount of urine creatinine.

Other experimental methods can be used to determine pathologic injury, apoptosis, and the expression of inflammatory markers in kidney samples. For example, immunohistochemistry, measurements of the expression of Tie2 phosphorylation or VE-PTP in tissue samples, and histological imaging can be used to determine pathologic injury, apoptosis, and the expression of inflammatory markers in kidney samples. In some embodiments, histological imaging can be used to detect apoptosis in kidney homogenate using, for example, DNA laddering or terminal deoxynucleotidyl transferase dUTP nick end labeling (TUNEL) staining. In some embodiments, RT-PCR can be used to determine the mRNA expression of mediators of inflammation and coagulation in kidney homogenate.

### EXAMPLES

### Example 1. Compounds with Inhibitory Activity to HPTP-ƒ3

Non-limiting examples of the HPTP-β IC₅₀ (μM) activity for illustrative compounds are listed in TABLE 1.

TABLE 1

| | | HPTP-β |
|---|---|---|
| No. | Compound | IC₅₀ μM |
| AA1 | 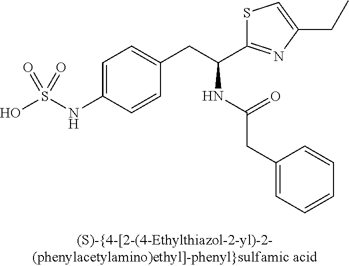 [SMILES: CCc1csc([C@H](Cc2ccc(NS(=O)(=O)O)cc2)NC(=O)Cc2ccccc2)n1] | 0.000157 |
| AA2 | 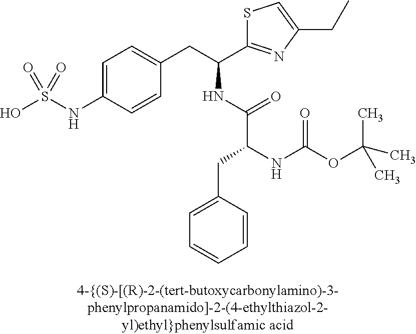 [SMILES: CCc1csc([C@H](Cc2ccc(NS(=O)(=O)O)cc2)NC(=O)[C@@H](Cc2ccccc2)NC(=O)OC(C)(C)C)n1] | 0.004 |
| AA3 | 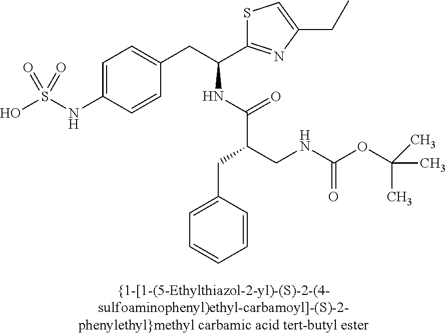 [SMILES: CCc1csc([C@H](Cc2ccc(NS(=O)(=O)O)cc2)NC(=O)[C@H](CNC(=O)OC(C)(C)C)Cc2ccccc2)n1] | 0.031 |
| AA4 | 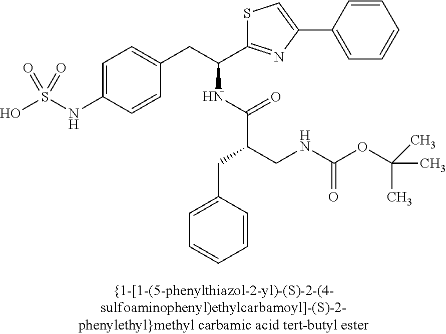 [SMILES: CC(C)(C)OC(=O)NC[C@H](Cc1ccccc1)C(=O)N[C@@H](Cc1ccc(NS(=O)(=O)O)cc1)c1nc(-c2ccccc2)cs1] | <5 × 10⁻⁸ |
| AA5 | 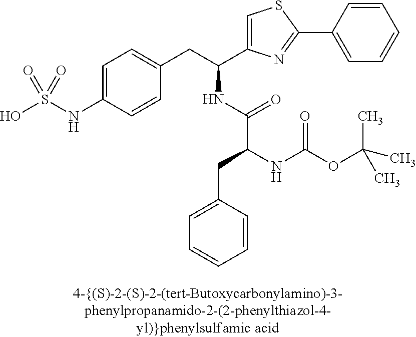 [SMILES: CC(C)(C)OC(=O)N[C@@H](Cc1ccccc1)C(=O)N[C@@H](Cc1ccc(NS(=O)(=O)O)cc1)c1csc(-c2ccccc2)n1] | <5 × 10⁻⁸ |
| AA6 | 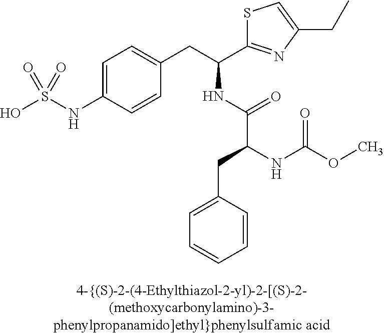 [SMILES: CCc1csc([C@H](Cc2ccc(NS(=O)(=O)O)cc2)NC(=O)[C@H](Cc2ccccc2)NC(=O)OC)n1] | 0.000162 |
| AA7 | 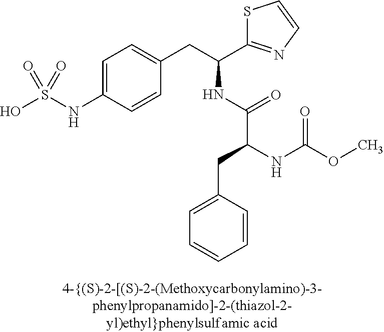 [SMILES: COC(=O)N[C@@H](Cc1ccccc1)C(=O)N[C@@H](Cc1ccc(NS(=O)(=O)O)cc1)c1nccs1] | 0.006 |
| AA8 | 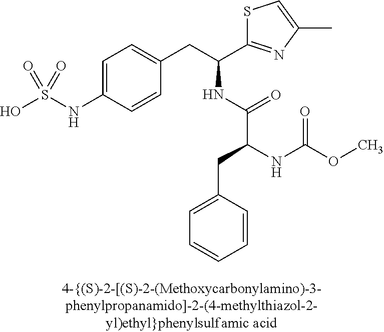 [SMILES: COC(=O)N[C@@H](Cc1ccccc1)C(=O)N[C@@H](Cc1ccc(NS(=O)(=O)O)cc1)c1nc(C)cs1] | 0.001 |
| AA9 | 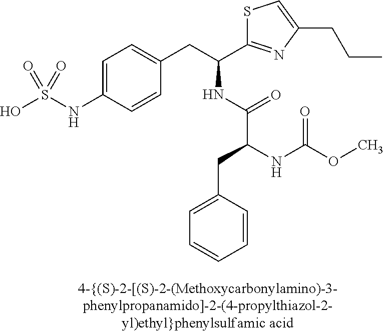 [SMILES: CCCc1csc([C@H](Cc2ccc(NS(=O)(=O)O)cc2)NC(=O)[C@H](Cc2ccccc2)NC(=O)OC)n1] | 0.0001 |
| AA10 | 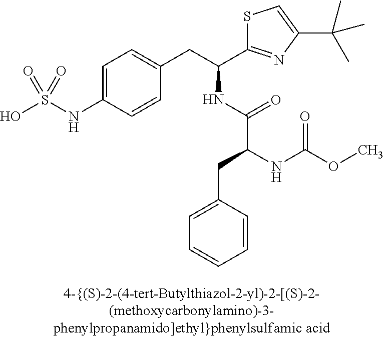 [SMILES: COC(=O)N[C@@H](Cc1ccccc1)C(=O)N[C@@H](Cc1ccc(NS(=O)(=O)O)cc1)c1nc(C(C)(C)C)cs1] | 0.0002 |
| AA11 | 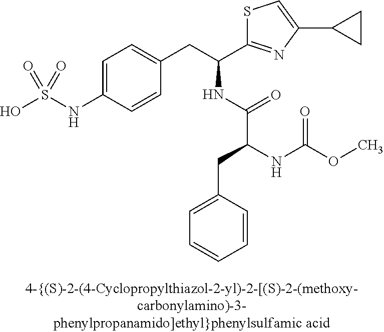 [SMILES: COC(=O)N[C@@H](Cc1ccccc1)C(=O)N[C@@H](Cc1ccc(NS(=O)(=O)O)cc1)c1nc(C2CC2)cs1] | 0.00001 |
| AA12 | 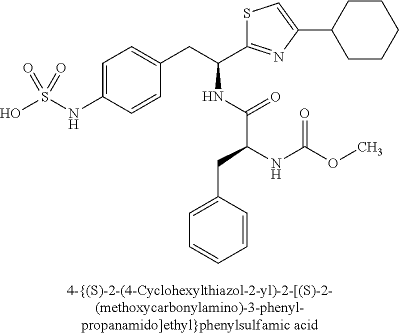 [SMILES: COC(=O)N[C@@H](Cc1ccccc1)C(=O)N[C@@H](Cc1ccc(NS(=O)(=O)O)cc1)c1nc(C2CCCCC2)cs1] | <5 × 10⁻⁸ |
| AA13 | 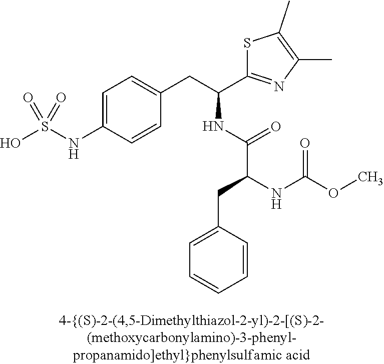 [SMILES: COC(=O)N[C@@H](Cc1ccccc1)C(=O)N[C@@H](Cc1ccc(NS(=O)(=O)O)cc1)c1nc(C)c(C)s1] | 0.001 |
| AA14 | 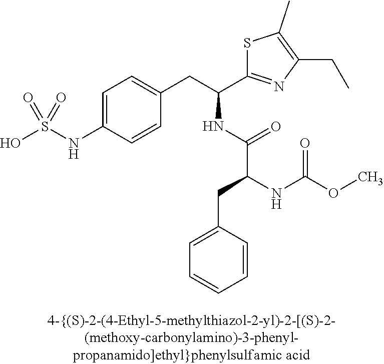 [SMILES: CCc1nc([C@H](Cc2ccc(NS(=O)(=O)O)cc2)NC(=O)[C@H](Cc2ccccc2)NC(=O)OC)sc1C] | 0.0001 |
| AA15 | 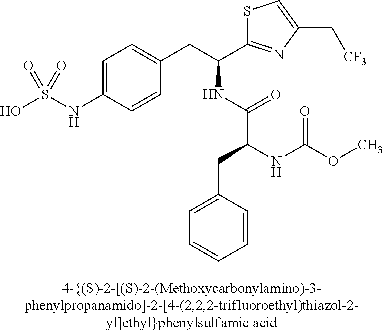 [SMILES: COC(=O)N[C@@H](Cc1ccccc1)C(=O)N[C@@H](Cc1ccc(NS(=O)(=O)O)cc1)c1nc(CC(F)(F)F)cs1] | 0.0003 |
| AA16 | 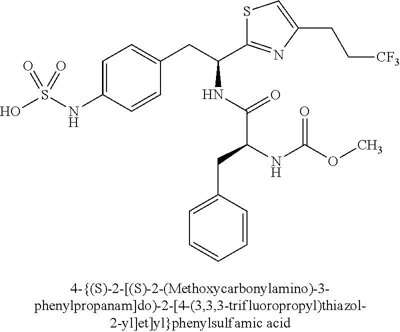 [SMILES: COC(=O)N[C@@H](Cc1ccccc1)C(=O)N[C@@H](Cc1ccc(NS(=O)(=O)O)cc1)c1nc(CCC(F)(F)F)cs1] | 0.00008 |
| AA17 | 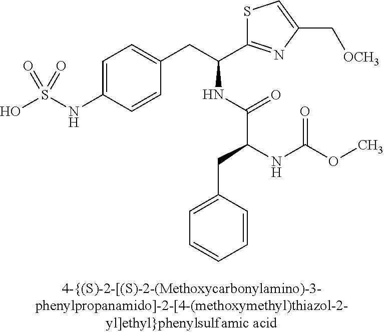 [SMILES: COCc1csc([C@H](Cc2ccc(NS(=O)(=O)O)cc2)NC(=O)[C@H](Cc2ccccc2)NC(=O)OC)n1] | 0.001 |
| AA18 | 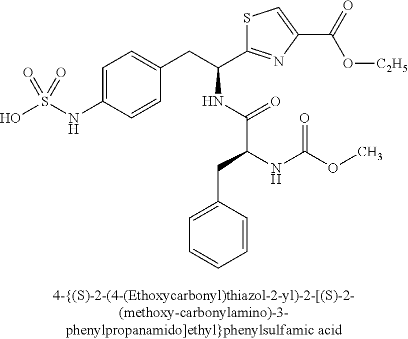 [SMILES: CCOC(=O)c1csc([C@H](Cc2ccc(NS(=O)(=O)O)cc2)NC(=O)[C@H](Cc2ccccc2)NC(=O)OC)n1] | 0.0002 |
| AA19 | 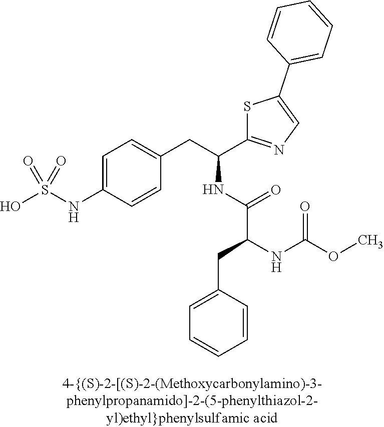 [SMILES: COC(=O)N[C@@H](Cc1ccccc1)C(=O)N[C@@H](Cc1ccc(NS(=O)(=O)O)cc1)c1ncc(-c2ccccc2)s1] | 0.0003 |
| AA20 | 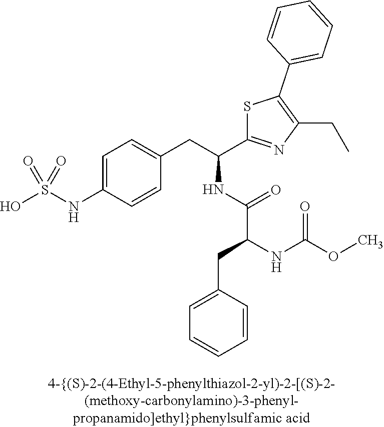 [SMILES: CCc1nc([C@H](Cc2ccc(NS(=O)(=O)O)cc2)NC(=O)[C@H](Cc2ccccc2)NC(=O)OC)sc1-c1ccccc1] | <5 × 10⁻⁸ |
| AA21 | 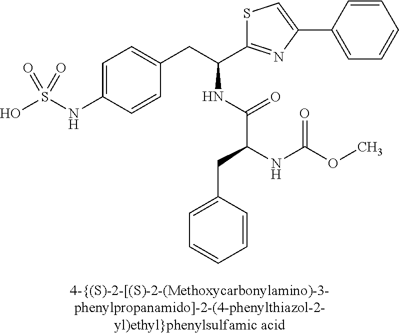 [SMILES: COC(=O)N[C@@H](Cc1ccccc1)C(=O)N[C@@H](Cc1ccc(NS(=O)(=O)O)cc1)c1nc(-c2ccccc2)cs1] | <2 × 10⁻⁶ |
| AA22 | 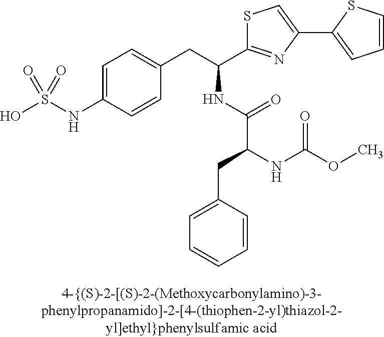 [SMILES: COC(=O)N[C@@H](Cc1ccccc1)C(=O)N[C@@H](Cc1ccc(NS(=O)(=O)O)cc1)c1nc(-c2cccs2)cs1] | <5 × 10⁻⁸ |
| AA23 | 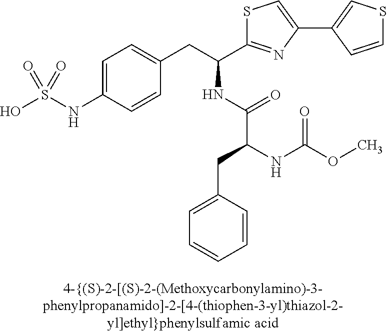 [SMILES: COC(=O)N[C@@H](Cc1ccccc1)C(=O)N[C@@H](Cc1ccc(NS(=O)(=O)O)cc1)c1nc(-c2ccsc2)cs1] | 0.00009 |
| AA24 | 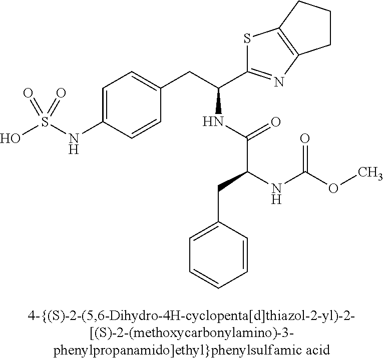 [SMILES: COC(=O)N[C@@H](Cc1ccccc1)C(=O)N[C@@H](Cc1ccc(NS(=O)(=O)O)cc1)c1nc2c(s1)CCC2] | 0.001 |
| AA25 | 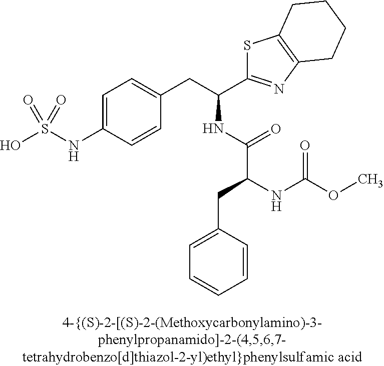 [SMILES: COC(=O)N[C@@H](Cc1ccccc1)C(=O)N[C@@H](Cc1ccc(NS(=O)(=O)O)cc1)c1nc2c(s1)CCCC2] | 0.0004 |
| AA26 | 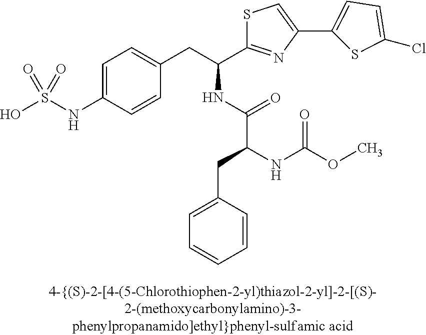 [SMILES: COC(=O)N[C@@H](Cc1ccccc1)C(=O)N[C@@H](Cc1ccc(NS(=O)(=O)O)cc1)c1nc(-c2ccc(Cl)s2)cs1] | <5 × 10⁻⁸ |
| AA27 | 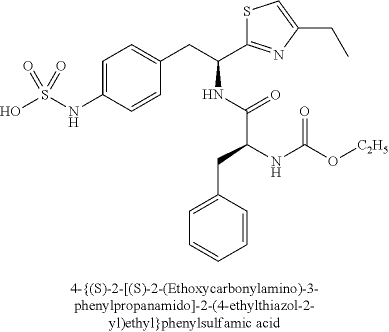 [SMILES: CCOC(=O)N[C@@H](Cc1ccccc1)C(=O)N[C@@H](Cc1ccc(NS(=O)(=O)O)cc1)c1nc(CC)cs1] | 0.00014 |
| AA28 | 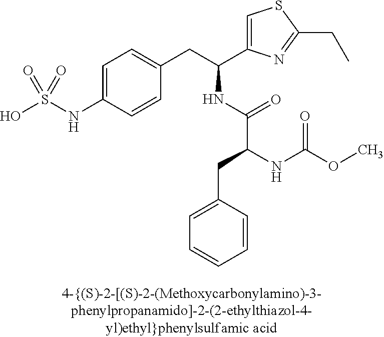 [SMILES: CCc1nc([C@H](Cc2ccc(NS(=O)(=O)O)cc2)NC(=O)[C@H](Cc2ccccc2)NC(=O)OC)cs1] | 0.0001 |
| AA29 | 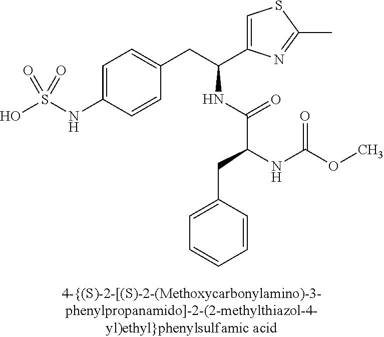 [SMILES: COC(=O)N[C@@H](Cc1ccccc1)C(=O)N[C@@H](Cc1ccc(NS(=O)(=O)O)cc1)c1csc(C)n1] | 0.001 |
| AA30 | 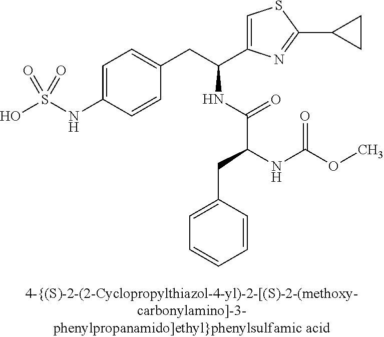 [SMILES: COC(=O)N[C@@H](Cc1ccccc1)C(=O)N[C@@H](Cc1ccc(NS(=O)(=O)O)cc1)c1csc(C2CC2)n1] | 0.0002 |
| AA31 | 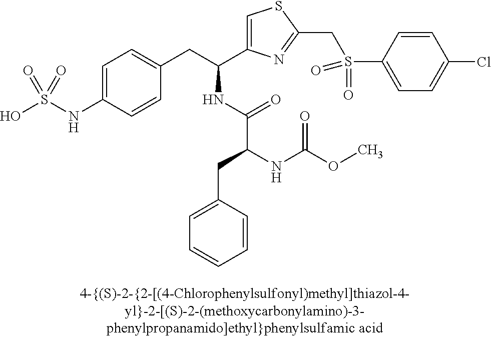 [SMILES: COC(=O)N[C@@H](Cc1ccccc1)C(=O)N[C@@H](Cc1ccc(NS(=O)(=O)O)cc1)c1csc(CS(=O)(=O)c2ccc(Cl)cc2)n1] | 0.00008 |
| AA32 | 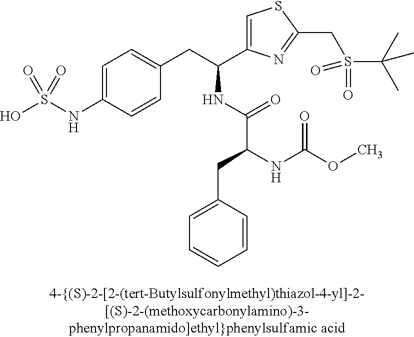 [SMILES: COC(=O)N[C@@H](Cc1ccccc1)C(=O)N[C@@H](Cc1ccc(NS(=O)(=O)O)cc1)c1csc(CS(=O)(=O)C(C)(C)C)n1] | 0.002 |
| AA33 | 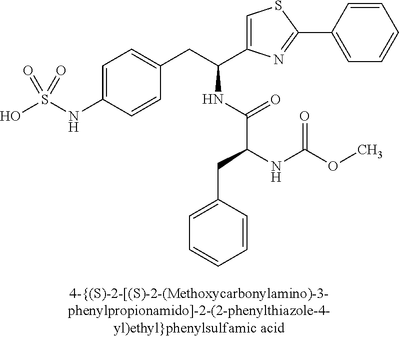 [SMILES: COC(=O)N[C@@H](Cc1ccccc1)C(=O)N[C@@H](Cc1ccc(NS(=O)(=O)O)cc1)c1csc(-c2ccccc2)n1] | 7 × 10⁻⁷ |
| AA34 | 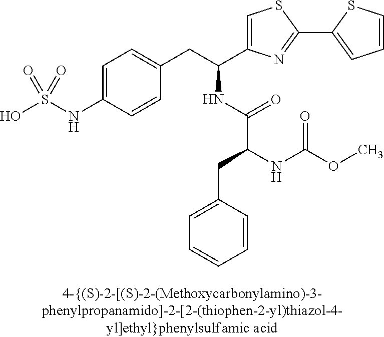 [SMILES: COC(=O)N[C@@H](Cc1ccccc1)C(=O)N[C@@H](Cc1ccc(NS(=O)(=O)O)cc1)c1csc(-c2cccs2)n1] | 5 × 10⁻⁸ |
| AA35 | 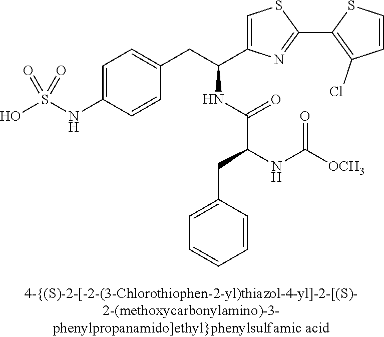 [SMILES: COC(=O)N[C@@H](Cc1ccccc1)C(=O)N[C@@H](Cc1ccc(NS(=O)(=O)O)cc1)c1csc(-c2sccc2Cl)n1] | <5 × 10⁻⁸ |
| AA36 | 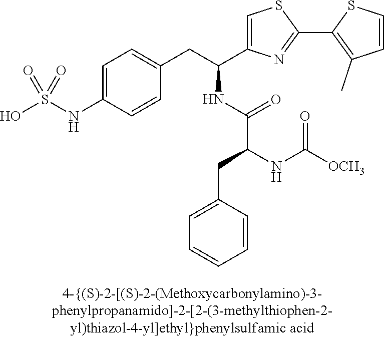 [SMILES: COC(=O)N[C@@H](Cc1ccccc1)C(=O)N[C@@H](Cc1ccc(NS(=O)(=O)O)cc1)c1csc(-c2sccc2C)n1] | <5 × 10⁻⁸ |
| AA37 | 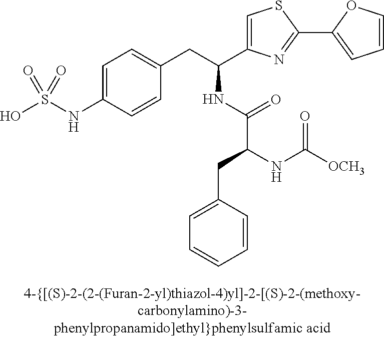 [SMILES: COC(=O)N[C@@H](Cc1ccccc1)C(=O)N[C@@H](Cc1ccc(NS(=O)(=O)O)cc1)c1csc(-c2ccco2)n1] | 0.0004 |
| AA38 | 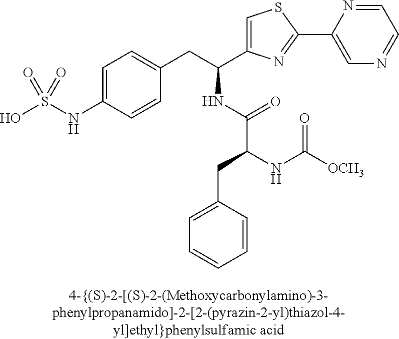 [SMILES: COC(=O)N[C@@H](Cc1ccccc1)C(=O)N[C@@H](Cc1ccc(NS(=O)(=O)O)cc1)c1csc(-c2cnccn2)n1] | 0.003 |
| AA39 | 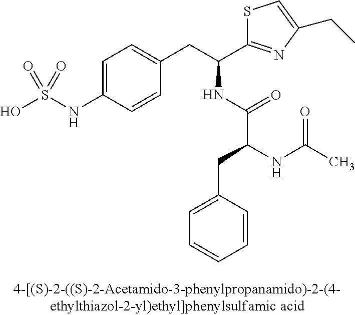 [SMILES: CCc1csc([C@H](Cc2ccc(NS(=O)(=O)O)cc2)NC(=O)[C@H](Cc2ccccc2)NC(C)=O)n1] | 0.001 |
| AA40 | 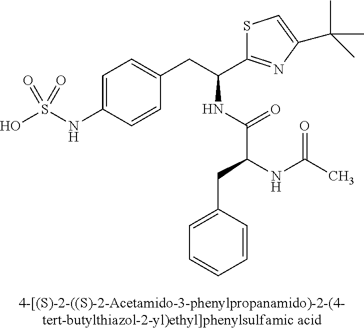 [SMILES: CC(=O)N[C@@H](Cc1ccccc1)C(=O)N[C@@H](Cc1ccc(NS(=O)(=O)O)cc1)c1nc(C(C)(C)C)cs1] | 0.0003 |
| AA41 | 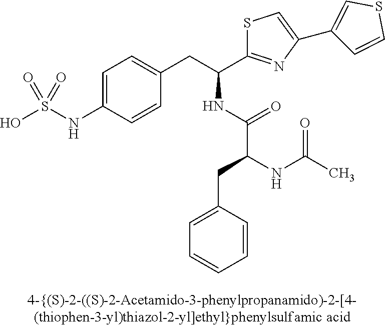 [SMILES: CC(=O)N[C@@H](Cc1ccccc1)C(=O)N[C@@H](Cc1ccc(NS(=O)(=O)O)cc1)c1nc(-c2ccsc2)cs1] | 0.00024 |
| AA42 | 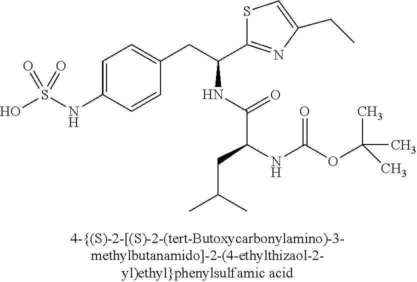 [SMILES: CCc1csc([C@H](Cc2ccc(NS(=O)(=O)O)cc2)NC(=O)[C@H](CC(C)C)NC(=O)OC(C)(C)C)n1] | 0.006 |
| AA43 | 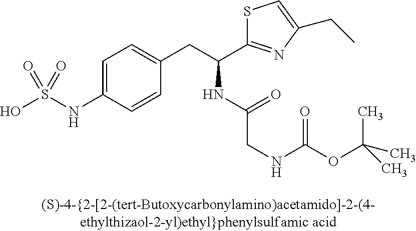 [SMILES: CCc1csc([C@H](Cc2ccc(NS(=O)(=O)O)cc2)NC(=O)CNC(=O)OC(C)(C)C)n1] | 0.028 |
| AA44 | 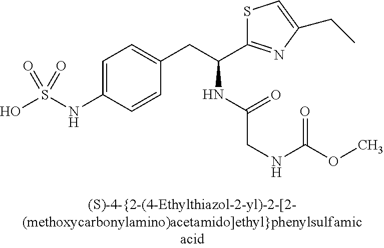 [SMILES: CCc1csc([C@H](Cc2ccc(NS(=O)(=O)O)cc2)NC(=O)CNC(=O)OC)n1] | 0.020 |
| AA45 | 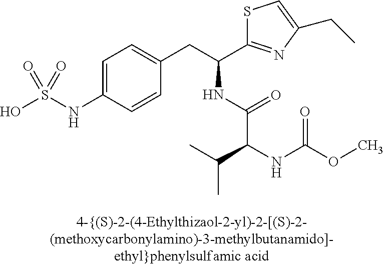 [SMILES: CCc1csc([C@H](Cc2ccc(NS(=O)(=O)O)cc2)NC(=O)[C@@H](NC(=O)OC)C(C)C)n1] | 0.003 |
| AA46 | 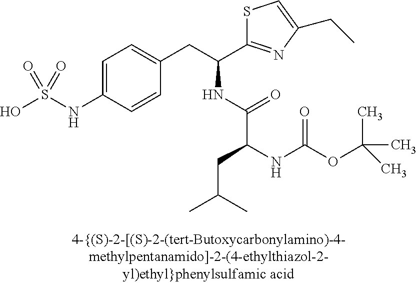 [SMILES: CCc1csc([C@H](Cc2ccc(NS(=O)(=O)O)cc2)NC(=O)[C@H](CC(C)C)NC(=O)OC(C)(C)C)n1] | 0.001 |
| AA47 | 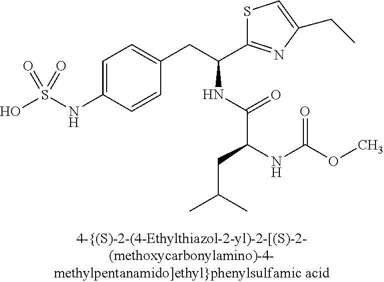 [SMILES: CCc1csc([C@H](Cc2ccc(NS(=O)(=O)O)cc2)NC(=O)[C@H](CC(C)C)NC(=O)OC)n1] | 0.0003 |
| AA48 | 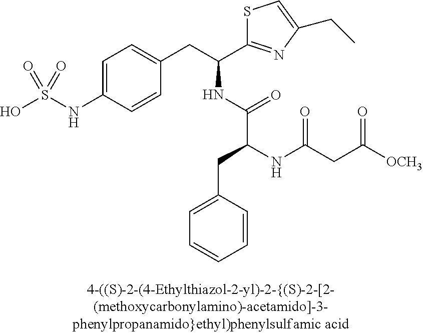 [SMILES: CCc1csc([C@H](Cc2ccc(NS(=O)(=O)O)cc2)NC(=O)[C@H](Cc2ccccc2)NC(=O)CC(=O)OC)n1] | 0.0003 |
| AA49 | 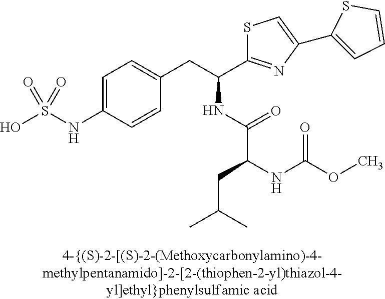 [SMILES: COC(=O)N[C@@H](CC(C)C)C(=O)N[C@@H](Cc1ccc(NS(=O)(=O)O)cc1)c1nc(-c2cccs2)cs1] | <5 × 10⁻⁸ |
| AA50 | 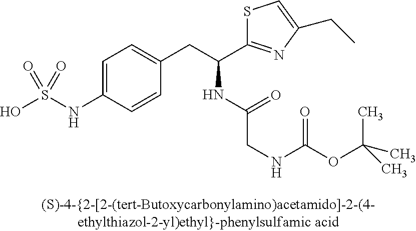 [SMILES: CCc1csc([C@H](Cc2ccc(NS(=O)(=O)O)cc2)NC(=O)CNC(=O)OC(C)(C)C)n1] | 0.028 |
| AA51 | 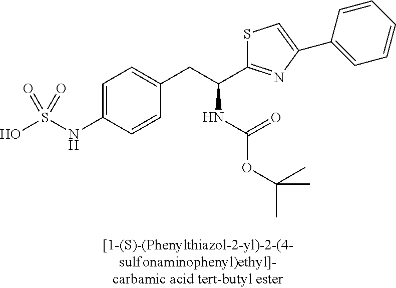 [SMILES: CC(C)(C)OC(=O)N[C@@H](Cc1ccc(NS(=O)(=O)O)cc1)c1nc(-c2ccccc2)cs1] | 0.049 |
| AA52 | 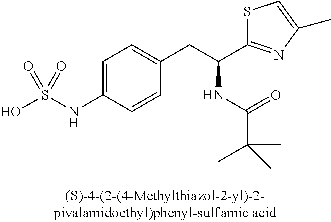 [SMILES: Cc1csc([C@H](Cc2ccc(NS(=O)(=O)O)cc2)NC(=O)C(C)(C)C)n1] | 0.112 |
| AA53 | 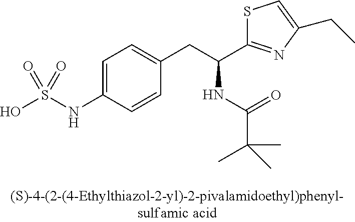 [SMILES: CCc1csc([C@H](Cc2ccc(NS(=O)(=O)O)cc2)NC(=O)C(C)(C)C)n1] | 0.085 |
| AA54 | 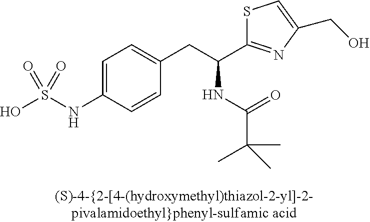 [SMILES: CC(C)(C)C(=O)N[C@@H](Cc1ccc(NS(=O)(=O)O)cc1)c1nc(CO)cs1] | 0.266 |
| AA55 | 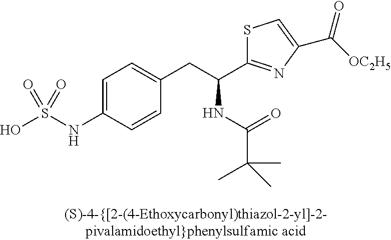 [SMILES: CCOC(=O)c1csc([C@H](Cc2ccc(NS(=O)(=O)O)cc2)NC(=O)C(C)(C)C)n1] | 0.584 |
| AA56 | 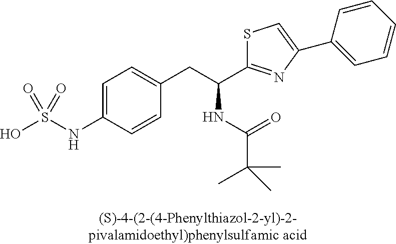 [SMILES: CC(C)(C)C(=O)N[C@@H](Cc1ccc(NS(=O)(=O)O)cc1)c1nc(-c2ccccc2)cs1] | 0.042 |
| AA57 | 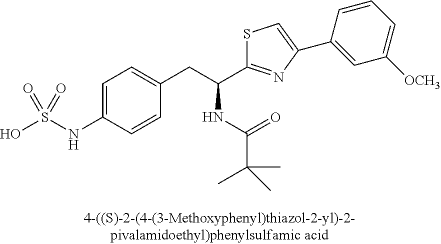 [SMILES: COc1cccc(-c2csc([C@H](Cc3ccc(NS(=O)(=O)O)cc3)NC(=O)C(C)(C)C)n2)c1] | 0.110 |
| AA58 | 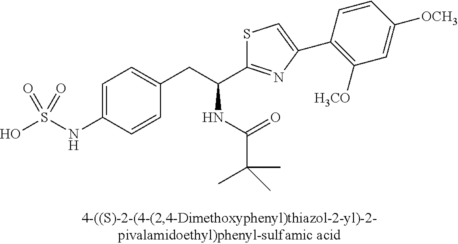 [SMILES: COc1ccc(-c2csc([C@H](Cc3ccc(NS(=O)(=O)O)cc3)NC(=O)C(C)(C)C)n2)c(OC)c1] | 0.086 |
| AA59 | 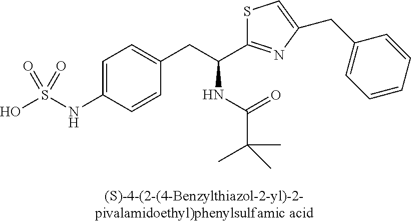 [SMILES: CC(C)(C)C(=O)N[C@@H](Cc1ccc(NS(=O)(=O)O)cc1)c1nc(Cc2ccccc2)cs1] | 0.113 |
| AA60 | 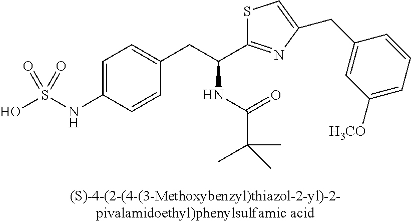 [SMILES: COc1cccc(Cc2csc([C@H](Cc3ccc(NS(=O)(=O)O)cc3)NC(=O)C(C)(C)C)n2)c1] | 0.132 |
| AA61 | 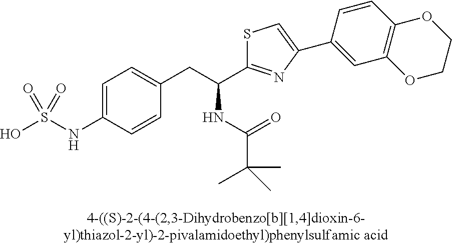 [SMILES: CC(C)(C)C(=O)N[C@@H](Cc1ccc(NS(=O)(=O)O)cc1)c1nc(-c2ccc3c(c2)OCCO3)cs1] | 0.138 |
| AA62 | 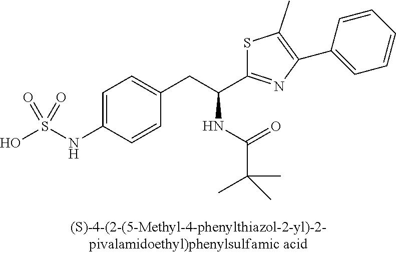 [SMILES: Cc1sc([C@H](Cc2ccc(NS(=O)(=O)O)cc2)NC(=O)C(C)(C)C)nc1-c1ccccc1] | 0.098 |
| AA63 | 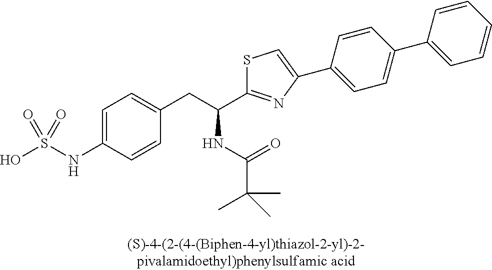 [SMILES: CC(C)(C)C(=O)N[C@@H](Cc1ccc(NS(=O)(=O)O)cc1)c1nc(-c2ccc(-c3ccccc3)cc2)cs1] | 0.381 |
| AA64 | 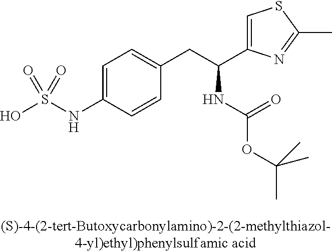 [SMILES: Cc1nc([C@H](Cc2ccc(NS(=O)(=O)O)cc2)NC(=O)OC(C)(C)C)cs1] | 0.033 |
| AA65 | 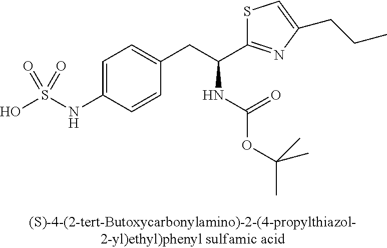 [SMILES: CCCc1csc([C@H](Cc2ccc(NS(=O)(=O)O)cc2)NC(=O)OC(C)(C)C)n1] | 0.04 |
| AA66 | 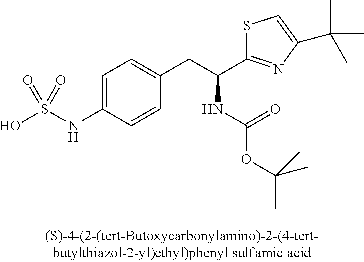 [SMILES: CC(C)(C)OC(=O)N[C@@H](Cc1ccc(NS(=O)(=O)O)cc1)c1nc(C(C)(C)C)cs1] | 0.027 |
| AA67 | 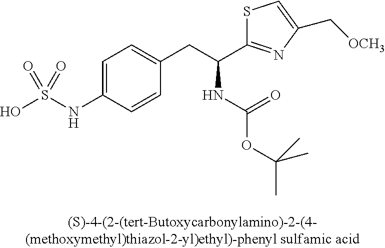 [SMILES: COCc1csc([C@H](Cc2ccc(NS(=O)(=O)O)cc2)NC(=O)OC(C)(C)C)n1] | 0.18 |
| AA68 | 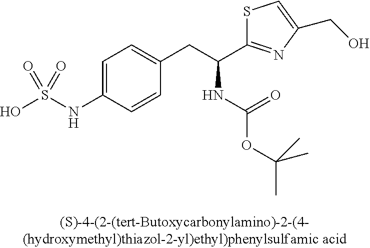 [SMILES: CC(C)(C)OC(=O)N[C@@H](Cc1ccc(NS(=O)(=O)O)cc1)c1nc(CO)cs1] | 0.644 |
| AA69 | 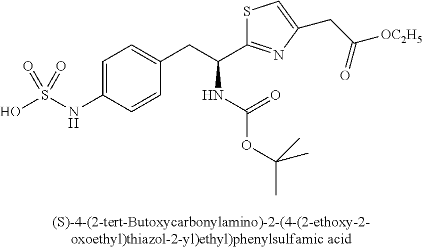 [SMILES: CCOC(=O)Cc1csc([C@H](Cc2ccc(NS(=O)(=O)O)cc2)NC(=O)OC(C)(C)C)n1] | 0.167 |
| AA70 | 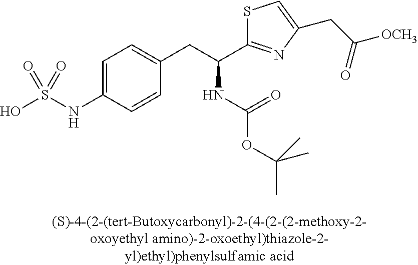 [SMILES: COC(=O)Cc1csc([C@H](Cc2ccc(NS(=O)(=O)O)cc2)NC(=O)OC(C)(C)C)n1] | 0.132 |
| AA71 | 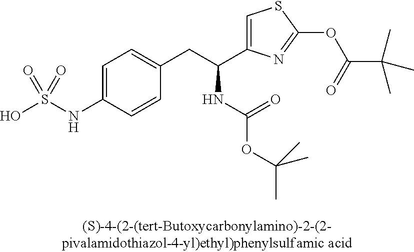 [SMILES: CC(C)(C)OC(=O)N[C@@H](Cc1ccc(NS(=O)(=O)O)cc1)c1csc(OC(=O)C(C)(C)C)n1] | 0.555 |
| AA72 | 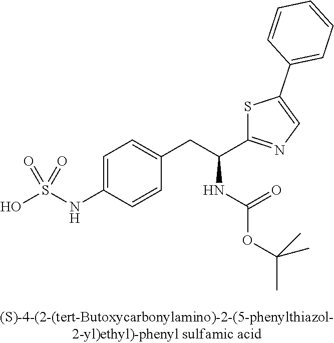 [SMILES: CC(C)(C)OC(=O)N[C@@H](Cc1ccc(NS(=O)(=O)O)cc1)c1ncc(-c2ccccc2)s1] | 0.308 |
| AA73 | 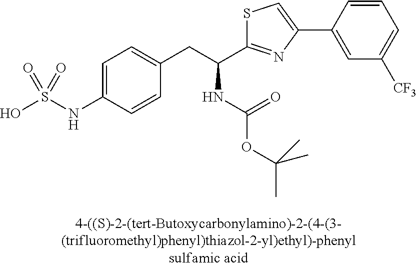 [SMILES: CC(C)(C)OC(=O)N[C@@H](Cc1ccc(NS(=O)(=O)O)cc1)c1nc(-c2cccc(C(F)(F)F)c2)cs1] | 0.253 |
| AA74 | 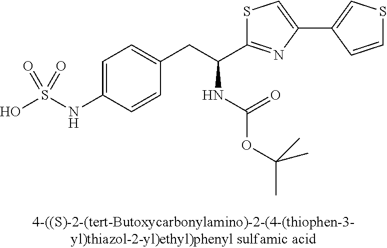 [SMILES: CC(C)(C)OC(=O)N[C@@H](Cc1ccc(NS(=O)(=O)O)cc1)c1nc(-c2ccsc2)cs1] | 0.045 |
| AA75 | 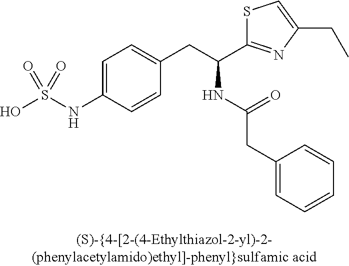 [SMILES: CCc1csc([C@H](Cc2ccc(NS(=O)(=O)O)cc2)NC(=O)Cc2ccccc2)n1] | 0.05 |
| AA76 | 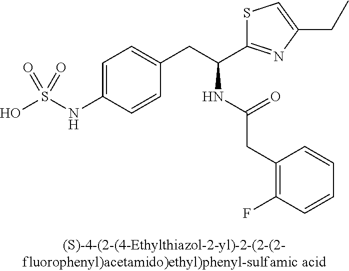 [SMILES: CCc1csc([C@H](Cc2ccc(NS(=O)(=O)O)cc2)NC(=O)Cc2ccccc2F)n1] | 0.012 |
| AA77 | 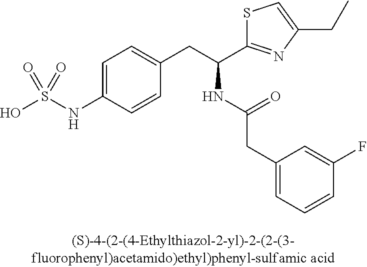 [SMILES: CCc1csc([C@H](Cc2ccc(NS(=O)(=O)O)cc2)NC(=O)Cc2cccc(F)c2)n1] | 0.0003 |
| AA78 | 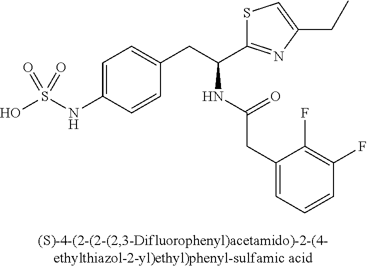 [SMILES: CCc1csc([C@H](Cc2ccc(NS(=O)(=O)O)cc2)NC(=O)Cc2cccc(F)c2F)n1] | 0.028 |
| AA79 | 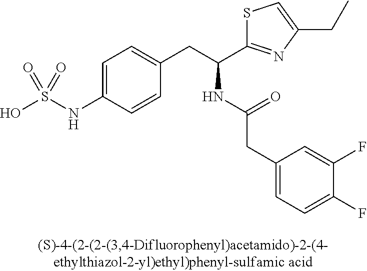 [SMILES: CCc1csc([C@H](Cc2ccc(NS(=O)(=O)O)cc2)NC(=O)Cc2ccc(F)c(F)c2)n1] | 0.075 |
| AA80 | 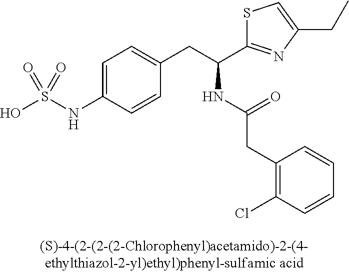 [SMILES: CCc1csc([C@H](Cc2ccc(NS(=O)(=O)O)cc2)NC(=O)Cc2ccccc2Cl)n1] | 0.056 |
| AA81 | 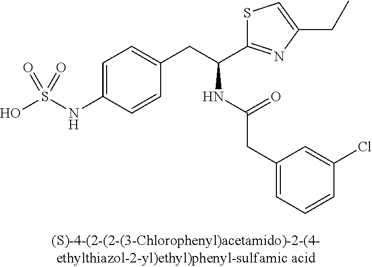 [SMILES: CCc1csc([C@H](Cc2ccc(NS(=O)(=O)O)cc2)NC(=O)Cc2cccc(Cl)c2)n1] | 0.033 |
| AA82 | 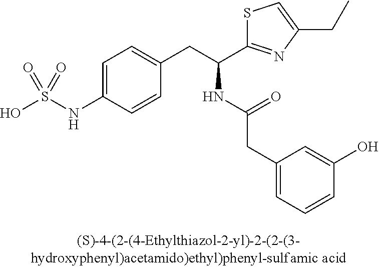 [SMILES: CCc1csc([C@H](Cc2ccc(NS(=O)(=O)O)cc2)NC(=O)Cc2cccc(O)c2)n1] | 0.04 |
| AA83 | 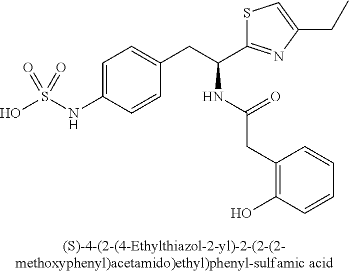 [SMILES: CCc1csc([C@H](Cc2ccc(NS(=O)(=O)O)cc2)NC(=O)Cc2ccccc2O)n1] | 0.014 |
| AA84 | 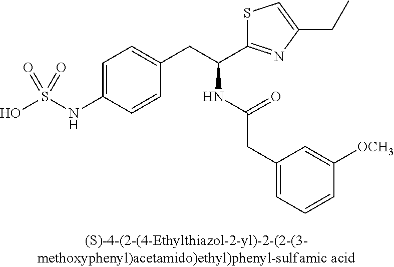 [SMILES: CCc1csc([C@H](Cc2ccc(NS(=O)(=O)O)cc2)NC(=O)Cc2cccc(OC)c2)n1] | 0.008 |
| AA85 | 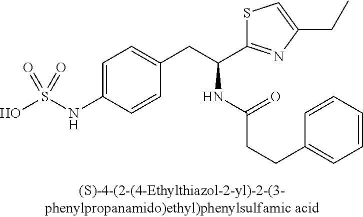 [SMILES: CCc1csc([C@H](Cc2ccc(NS(=O)(=O)O)cc2)NC(=O)CCc2ccccc2)n1] | 0.002 |
| AA86 | 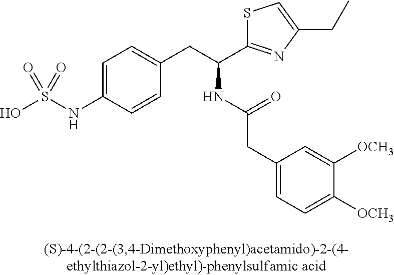 [SMILES: CCc1csc([C@H](Cc2ccc(NS(=O)(=O)O)cc2)NC(=O)Cc2ccc(OC)c(OC)c2)n1] | 0.028 |
| AA87 | 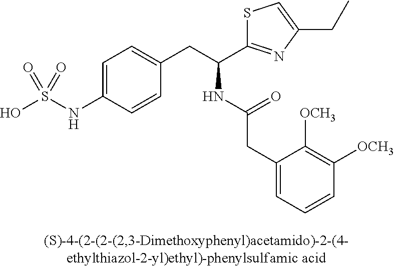 [SMILES: CCc1csc([C@H](Cc2ccc(NS(=O)(=O)O)cc2)NC(=O)Cc2cccc(OC)c2OC)n1] | 0.037 |
| AA88 | 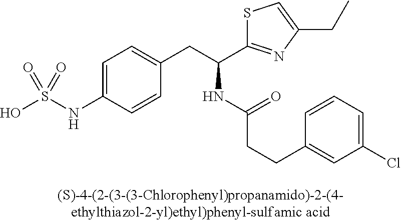 [SMILES: CCc1csc([C@H](Cc2ccc(NS(=O)(=O)O)cc2)NC(=O)CCc2cccc(Cl)c2)n1] | 0.0002 |
| AA89 | 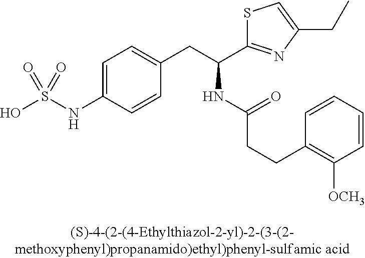 [SMILES: CCc1csc([C@H](Cc2ccc(NS(=O)(=O)O)cc2)NC(=O)CCc2ccccc2OC)n1] | 0.003 |
| AA90 | 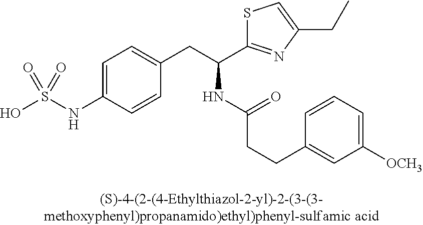 [SMILES: CCc1csc([C@H](Cc2ccc(NS(=O)(=O)O)cc2)NC(=O)CCc2cccc(OC)c2)n1] | 0.01 |
| AA91 | 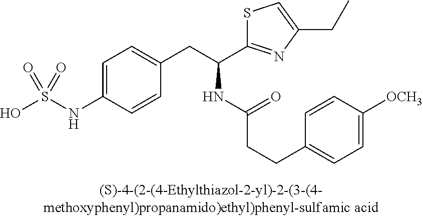 [SMILES: CCc1csc([C@H](Cc2ccc(NS(=O)(=O)O)cc2)NC(=O)CCc2ccc(OC)cc2)n1] | 0.006 |
| AA92 | 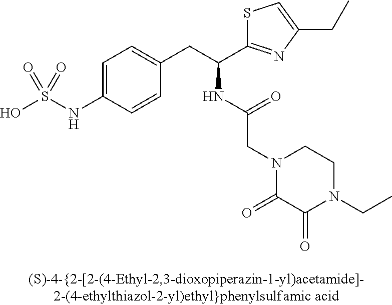 [SMILES: CCc1csc([C@H](Cc2ccc(NS(=O)(=O)O)cc2)NC(=O)CN2CCN(CC)C(=O)C2=O)n1] | 0.002 |
| AA93 | 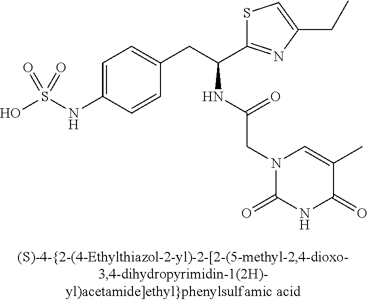 [SMILES: CCc1csc([C@H](Cc2ccc(NS(=O)(=O)O)cc2)NC(=O)Cn2cc(C)c(=O)[nH]c2=O)n1] | 0.002 |
| AA94 | 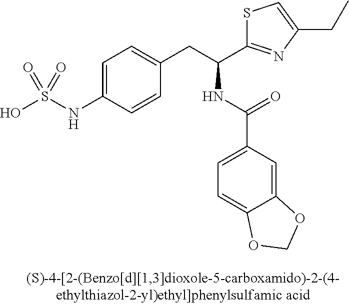 [SMILES: CCc1csc([C@H](Cc2ccc(NS(=O)(=O)O)cc2)NC(=O)c2ccc3c(c2)OCO3)n1] | 0.042 |
| AA95 | 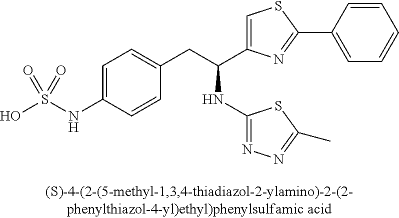 [SMILES: Cc1nnc(N[C@@H](Cc2ccc(NS(=O)(=O)O)cc2)c2csc(-c3ccccc3)n2)s1] | 0.003 |
| AA96 | 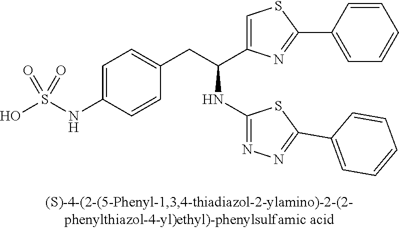 [SMILES: O=S(=O)(O)Nc1ccc(C[C@H](Nc2nnc(-c3ccccc3)s2)c2csc(-c3ccccc3)n2)cc1] | 0.046 |
| AA97 | 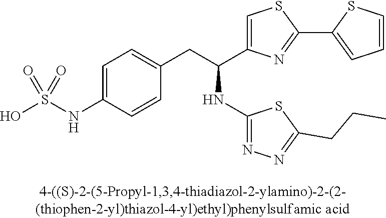 [SMILES: CCCc1nnc(N[C@@H](Cc2ccc(NS(=O)(=O)O)cc2)c2csc(-c3cccs3)n2)s1] | 0.0002 |
| AA98 | 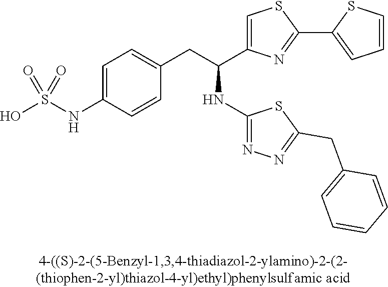 [SMILES: O=S(=O)(O)Nc1ccc(C[C@H](Nc2nnc(Cc3ccccc3)s2)c2csc(-c3cccs3)n2)cc1] | 0.0006 |
| AA99 | 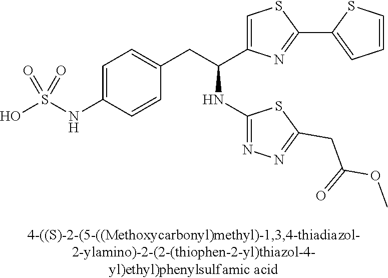 [SMILES: COC(=O)Cc1nnc(N[C@@H](Cc2ccc(NS(=O)(=O)O)cc2)c2csc(-c3cccs3)n2)s1] | 0.002 |
| AA100 | 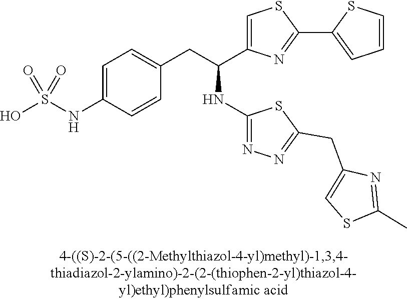 [SMILES: Cc1nc(Cc2nnc(N[C@@H](Cc3ccc(NS(=O)(=O)O)cc3)c3csc(-c4cccs4)n3)s2)cs1] | 9 × 10⁻⁶ |

### Example 2. Assessment of Renal Function Indices in Subjects Treated with a Compound Disclosed Herein

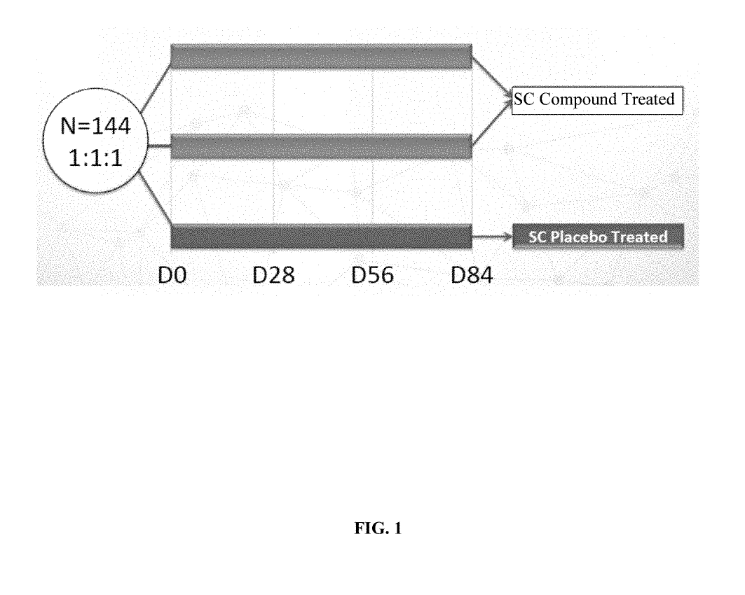

A set-up of a study to test the effect of compound AA34 in TABLE 1 is shown in FIG. 1.

The human subjects treated with compound AA34 were administered 15 mg of the compound subcutaneously twice a day for three months. One group of subjects received the compound and ranibizumab (RBZ; 0.3 mg monthly via intraocular injection), while the other group received only the compound with a sham intraocular injection. The subjects in the placebo arm received a placebo subcutaneously twice a day along with ranibizumab. Among the subjects in the study, 50% did not have albuminuria at baseline (<30 mg/g UACR) and 50% had albuminuria (>30 mg/g UACR). Within the subjects that had albuminuria, 30% had microalbuminuria (>30 mg/g and <300 mg/g UACR) and 20% had macroalbuminuria (>300 mg/g UACR).

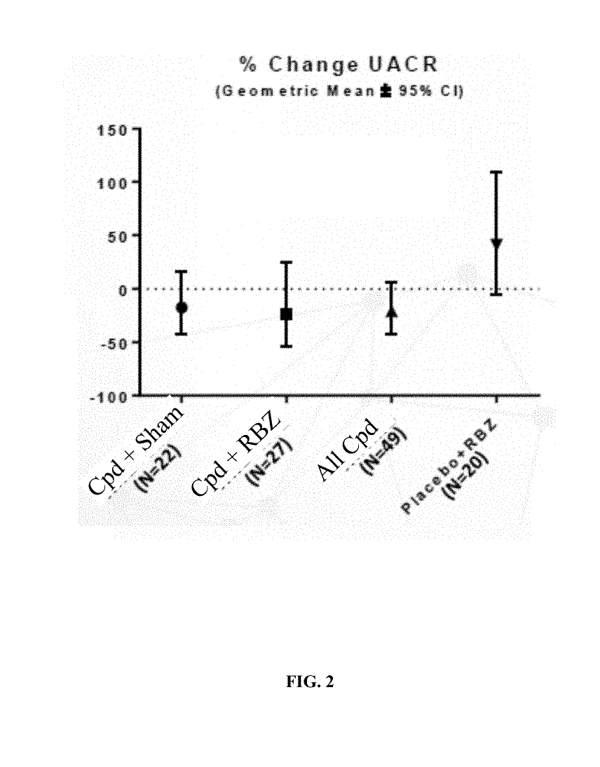

The results of the study are shown in FIG. 2 and TABLE 2 as the % change from baseline to end of treatment (three months) in geometric mean +95% confidence interval in the patients with albuminuria at baseline.

TABLE 2

| | | Compound + | All | Placebo + |
|---|---|---|---|---|
| | Compound | RBZ | Compound | RBZ |
| n | 22 | 27 | 49 | 20 |
| % Change in | −17.7 | −23.9 | −21.2 | 41.1 |
| Geometric Mean | | | | |
| p-value, unadjusted | | | 0.03 | |
| (Cpd v. Placebo) | | | | |
| p-value, ANCOVA* | | | 0.006 | |
| (Cpd v. Placebo) | | | | |

*Adjusted for standard variabes (baseline UACR, Hemoglobin A1c (HgbA1c), systolic blood pressure)

There was approximately a 20% reduction in the UACR in patients receiving compound AA34 compared to the UACR in patients receiving placebo. The results indicated that albuminuric and microalbuminuric diabetic subjects treated for three months with compound AA34 had a statistically significantly greater percentage change from baseline in geometric mean UACR compared to placebo treated subjects (p=0.03 and 0.001, respectively). The statistical significance was maintained or strengthened when standard baseline covariates (UACR, HgbAIc, and systolic blood pressure) were controlled for in the models (p=0.005 and 0.002, respectively).

Statistical analysis using ANCOVA is shown below in TABLE 3:

TABLE 3
Statistical Analysis: p-values for Different Models for Change in
Natural Log-transformed UACR
Comparison: All Compound groups v. Placebo + RBZ

| | | | | | Model |
|---|---|---|---|---|---|
| | | | | Model | Terms: |
| | | | Model | Terms: | Log(base- |
| | | | Terms: | Log(base- | line |
| | | Model | Log(base- | line | UACR) |
| | | Terms: | line | UACR) | Baseline |
| | | Log(base- | UACR) | Baseline | HGBAIC |
| | | line | Baseline | systolic | Baseline |
| Population | t-test | UACR) | HGBAIC | BP | systolic BP |
| All Subjects | 0.0661 | 0.1162 | 0.1267 | 0.1214 | 0.1346 |
| Baseline | 0.0317 | 0.0040^{a} | 0.0041^{a} | 0.0053^{a} | 0.0056^{a} |
| UACR >30 | | | | | |
| Baseline | 0.0012 | 0.0012 | 0.0014 | 0.0014 | 0.0017 |
| UACR 30- | | | | | |
| 300 | | | | | |

All transformations are natural log transformations. All models include term for treatment. p-values in table are from test comparing change in natural log-transformed UACR between treatment groups.
^{a}includes interaction term (natural log of baseline UACR by treatment) due to statistical significance (p = 0.0158)

TABLE 4 below provides a shift analysis of the results of the study:

TABLE 4

| | Baseline | | | | |
|---|---|---|---|---|---|
| | UACR | | Month 3/EOT | | |
| Treatment | (mg/g) | n | <30 | 30-300 | >300 |
| All compound | | 92 | | | |
| | <30 | 43 | 41 (95%) | 2 (5%) | 0 |
| | 30-300 | 31 | 4 (13%) | 25 (81%) | 2 (6%) |
| | >300 | 18 | 1 (6%) | 1 (6%) | 16 (89%) |
| Pbo | | 46 | | | |
| | <30 | 26 | 26 (100%) | 0 | 0 |
| | 30-300 | 11 | 0 | 7 (64%) | 4 (36%) |
| | >300 | 9 | 0 | 0 | 9 (100%) |

### Example 3. Phosphatase Inhibition Protects Against Endotoxin-Induced Acute Kidney Injury

Male C57BL6 mice, 9-10 weeks old, were injected i.p. with 0.2 mg E. coli LPS per 25 g body weight. The mice were injected with compound AA34 (50 mg/kg, 50 μL) or the vehicle (50 μL) at the time of lipopolysaccharide (LPS) injection, 8 after LPS injection, and 16 hours after LPS injection. The mice were sacrificed 24 h after the LPS injection. The saline-injected mice were studied in parallel as controls.

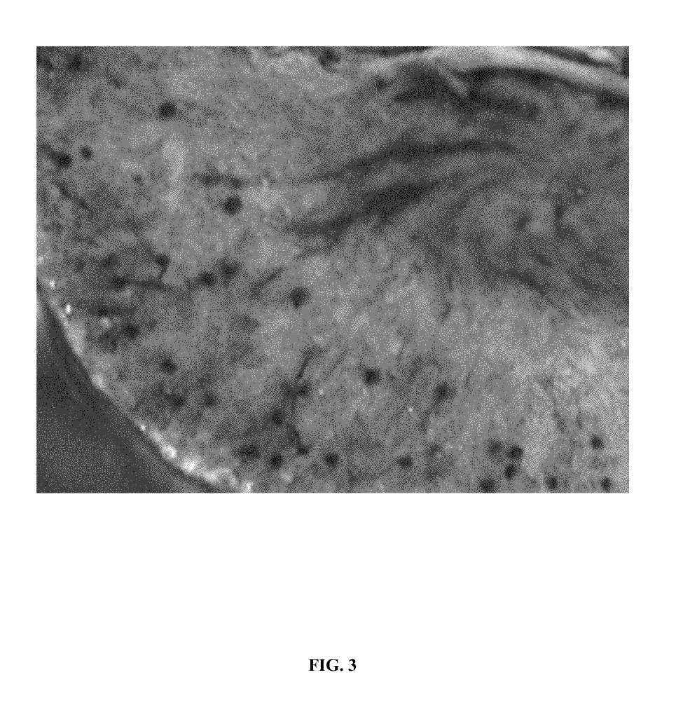

FIG. 3 shows the expression VE-PTP expressed in kidney tissue endothelium.

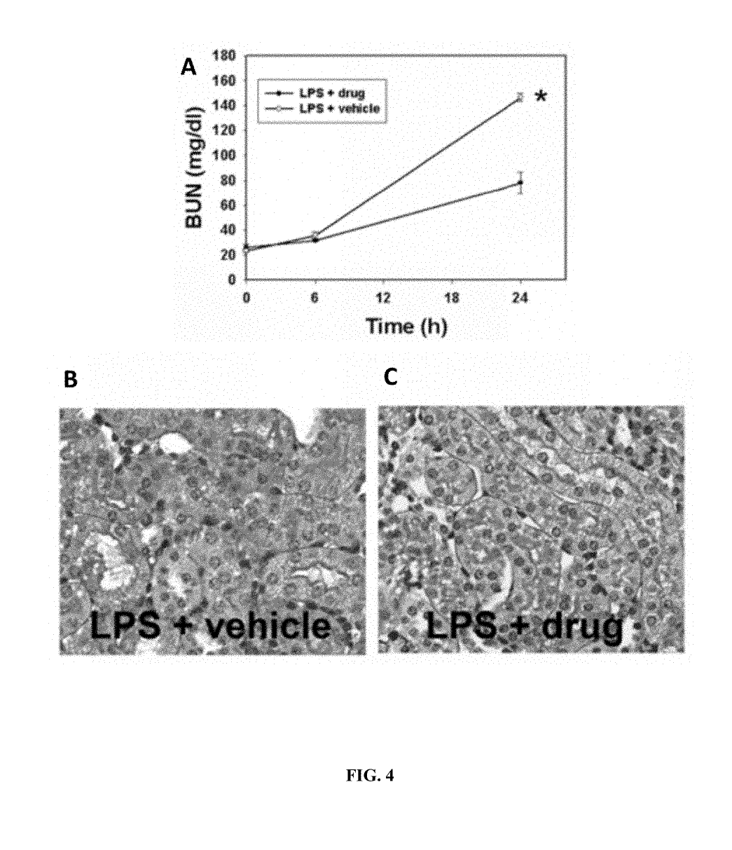

Compound AA34 protected mice against acute kidney injury, as shown in FIG. 4. Serum samples from the mice were analyzed for BUN as a marker of kidney function. FIG. 4 PANEL A shows that LPS caused an abrupt rise in BUN, which was statistically reduced in mice treated with compound AA34 (*p=0.002). The histological results showed less morphologic tubular injury in the drug versus the vehicle groups 24 h after LPS injection. PANEL B shows a histological image obtained from a mouse treated with LPS+vehicle. PANEL C shows a histological image obtained from a mouse treated with LPS+Compound AA34. The data show that treatment with compound AA34 reduced the rise in BUN and reduced morphologic tubular injury after the mice received LPS injections, and thus that compound AA34 was protective against acute kidney injury.

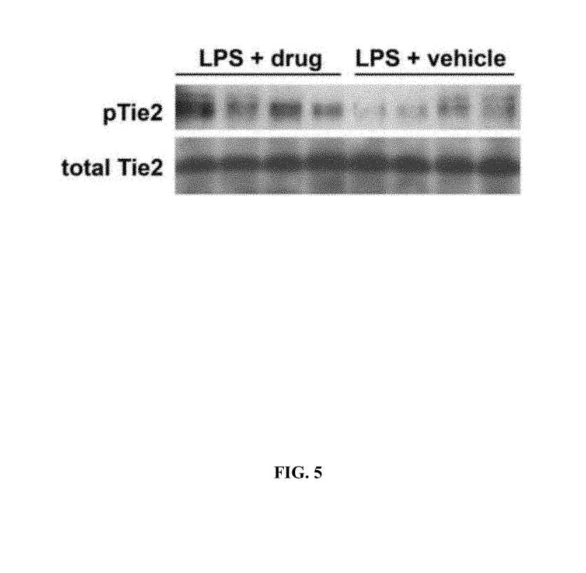

Compound AA34 preserved Tie2 phosphorylation, as shown in FIG. 5. Immunoprecipitation of kidney homogenate was performed to analyze the extent of Tie2 phosphorylation in the drug versus vehicle groups. Immunoprecipitation of kidney homogenate was completed using anti-Tie2 Ab, which was probed using anti-phosphotyrosine Ab. Total Tie2 levels were equal in the drug-treated and vehicle-treated groups after being injected with LPS. The amount of tyrosine phosphorylated Tie2 was greater in the drug group, and the increased levels of tyrosine phosphorylated Tie2 persisted 8 h after the last dose of phosphatase inhibitor. FIG. 5 shows that the group treated with LPS+Compound AA34 exhibited increased levels of tyrosine phosphorylated Tie2 compared to the group treated with LPS+vehicle.

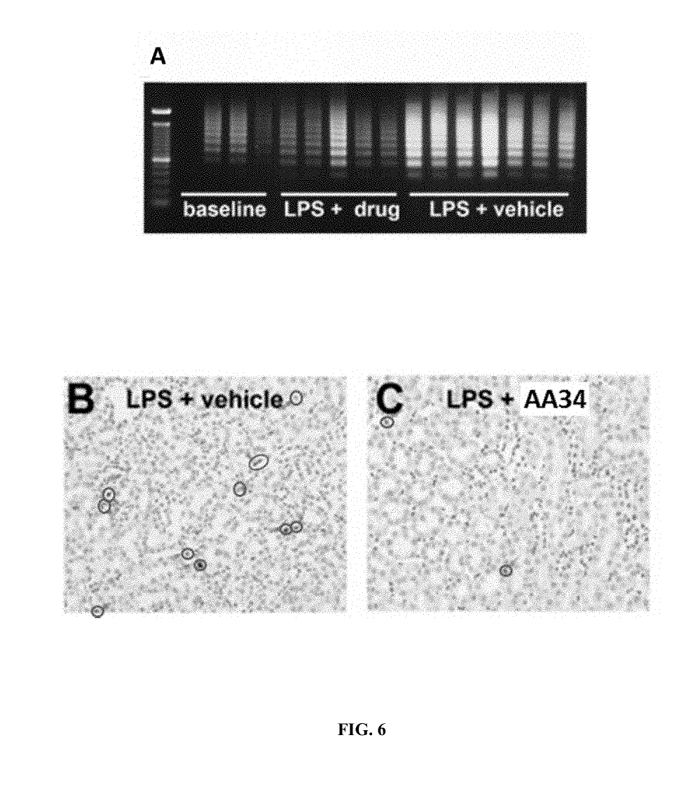

Compound AA34 reduced renal apoptosis, as shown in FIG. 6. Apoptosis was detected in kidney homogenate using DNA laddering, which is the result of apoptotic DNA fragmentation. FIG. 6 PANEL A shows DNA laddering used to detect apoptosis in kidney homogenate; each lane represents a different animal. In the lanes treated with compound AA34, less DNA laddering was visible due to the reduced apoptosis. Terminal deoxynucleotidyl transferase dUTP nick end labeling (TUNEL) staining was used to visualize the presence of apoptotic nuclei. PANEL B shows TUNEL staining of kidney homogenate obtained from animals treated with LPS+vehicle. PANEL C shows TUNEL staining of kidney homogenate obtained from animals treated with LPS+compound AA34. The data show that many of the apoptotic cells (circled) were present in the peritubular region, and the apoptotic cells were likely to be peritubular capillary endothelial cells. Overall, the results demonstrated that cells from animals treated with compound AA34 displayed less apoptosis.

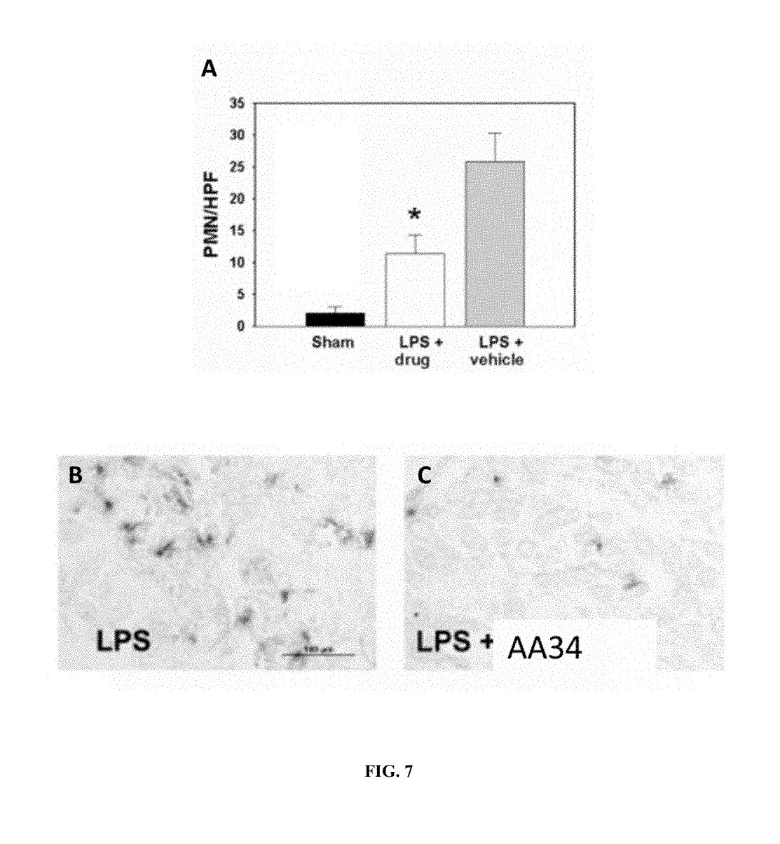

Phosphatase inhibition reduced LPS-induced renal neutrophil infiltration, as shown in FIG. 7. Phosphatase inhibition with compound AA34 significantly reduced renal neutrophil infiltration 24 h after LPS injection (*p<0.05 versus both groups). FIG. 7 PANEL A shows that treatment with compound AA34 reduced renal neutrophil infiltration by over 50% 24 h after LPS injection. PANEL B shows LPS-induced renal neutrophil infiltration in animals treated with LPS+vehicle. PANEL C shows LPS-induced renal neutrophil infiltration in animals treated with LPS+compound AA34. The images show that treatment with compound AA34 reduced renal neutrophil infiltration.

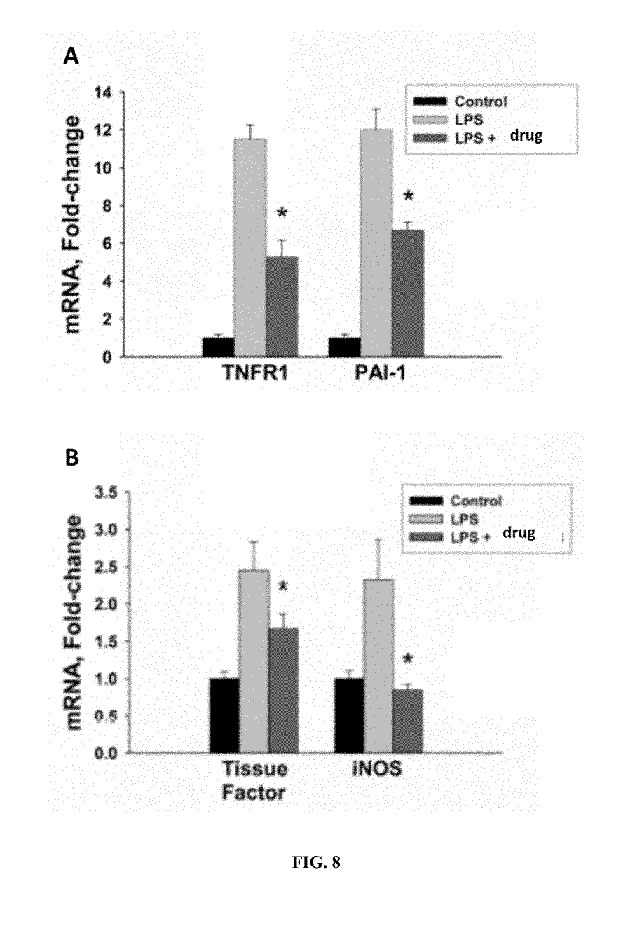

Phosphatase inhibition by compound AA34 reduced renal mRNA expression of various mediators of inflammation and coagulation, as shown in FIG. 8. The kidneys of the mice were analyzed for RNA level expression of various inflammatory mediators. RT-PCR of kidney homogenate showed that LPS induced increased expression of TNFR1, plasminogen activator inhibitor-1 (PAI-1), tissue factor, and inducible nitric oxide synthase (iNOS). The upregulation by LPS was significantly reduced in the drug-treated group (*p<0.05). TNFR1, PAI-1, and tissue factor were expressed mainly in the endothelium, the site of Tie2 expression. These data were consistent with an effect specific to the endothelium when Tie2 phosphorylation was preserved by compound AA34. FIG. 8 PANEL A shows that phosphatase inhibition by compound AA34 reduced renal mRNA expression of TNFR1 and PAI-1 by about 6-fold and about 5-fold, respectively. PANEL B shows that phosphatase inhibition by compound AA34 reduced renal mRNA expression of tissue factor and iNOS by about 0.8-fold and about 1.5-fold, respectively. The results indicated that treatment with compound AA34 reduced the expression of inflammatory and coagulation markers.

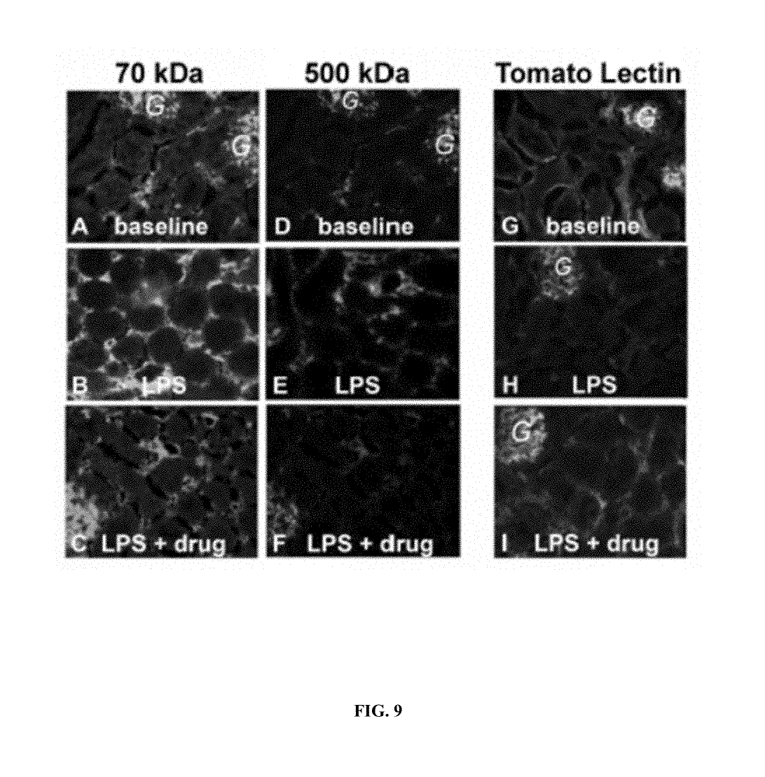

Phosphatase inhibition with compound AA34 reduced renal vascular leak observed in sepsis, as shown in FIG. 9. Mice were injected with LPS and drug (compound AA34) versus the vehicle (LPS only). 2 min prior to sacrificing the animals at 24 h, the mice were injected with 70 kDa and 500 kDa fluorescent fixable dextrans by intravenous catheter. Frozen sections showed that extrusion of dye beyond the small peritubular capillaries was induced by LPS, which was markedly reduced by treatment with compound AA34. Vascular integrity, shown by endothelial staining with tomato lectin, was markedly reduced after LPS injection. The extent of vascular injury was reduced by compound AA34, with relatively preserved tomato lectin staining of peritubular capillaries. FIG. 9 PANEL A shows a baseline image of a mouse injected with 70 kDa fluorescent fixable dextrans. PANEL B shows a fluorescence image of a mouse injected with LPS and subsequently injected with 70 kDa fluorescent fixable dextrans. PANEL C shows a fluorescence image of a mouse injected with LPS+drug, and subsequently injected with 70 kDa fluorescent fixable dextrans. PANEL D shows a baseline image of a mouse injected with 500 kDa fluorescent fixable dextrans. PANEL E shows a fluorescence image of a mouse injected with LPS and subsequently injected with 500 kDa fluorescent fixable dextrans. PANEL F shows a fluorescence image of a mouse injected with LPS+drug, and subsequently injected with 500 kDa fluorescent fixable dextrans. The data show that treatment with LPS and compound AA34 reduced the extent of vascular injury when imaged using 70 kDa and 500 kDa fluorescent fixable dextrans. PANEL G shows a baseline image of a mouse injected with tomato lectin. PANEL H shows a fluorescence image of a mouse injected with LPS and subsequently injected with tomato lectin. PANEL I shows a fluorescence image of a mouse injected with LPS+drug, and subsequently injected with tomato lectin. The data show that treatment with LPS and compound AA34 did not have a significant effect on the peritubular capillaries of the samples based on images obtained using tomato lectin staining.

Phosphatase inhibition using compound AA34 was successful in preserving renal endothelial Tie2 phosphorylation in septic acute kidney injury. Phosphatase inhibition using compound AA34 was associated with reduced LPS-induced renal inflammation, apoptosis, coagulation, and vascular leak. The effects of the pathways resulted in less renal dysfunction after LPS administration.

### EMBODIMENTS

The following non-limiting embodiments provide illustrative examples of the invention, but do not limit the scope of the invention.

### Embodiment 1

A method of treating nephropathy in a subject in need thereof, the method comprising administering to the subject a therapeutically-effective amount of a compound that activates Tie-2.

### Embodiment 2

The method of embodiment 1, wherein the compound has the formula:

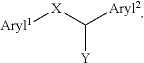

wherein:
Aryl¹ is an aryl group which is substituted or unsubstituted;
Aryl² is an aryl group which is substituted or unsubstituted;
X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

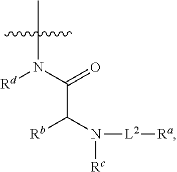

wherein:
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{c} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted,
   or a pharmaceutically-acceptable salt thereof, tautomer, or zwitterion thereof.

### Embodiment 3

The method of embodiment 2, wherein:
Aryl¹ is substituted or unsubstituted phenyl;
Aryl² is substituted or unsubstituted heteroaryl; and
X is alkylene.

### Embodiment 4

The method of embodiment 2 or 3, wherein:
Aryl¹ is substituted phenyl;
Aryl² is substituted heteroaryl; and
X is methylene.

### Embodiment 5

The method of any one of embodiments 2-4, wherein the compound that activates Tie-2 is a compound of the formula:

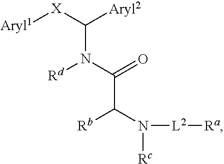

wherein
Aryl¹ is para-substituted phenyl;
Aryl² is substituted heteroaryl;
X is methylene;
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond;
R^{a} is H, alkyl, alkenyl, alkynyl, aryl, arylalkyl, heterocyclyl, heterocyclylalkyl, heteroaryl, or heteroarylalkyl, any of which is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted;
R^{c} is H or alkyl which is substituted or unsubstituted; and
R^{d} is H or alkyl which is substituted or unsubstituted.

### Embodiment 6

The method of any one of embodiments 2-5, wherein:
Aryl² is a substituted thiazole moiety;
L² together with the nitrogen atom to which L² is bound forms a carbamate linkage;
R^{a} is alkyl, which is substituted or unsubstituted;
R^{b} is arylalkyl, which is substituted or unsubstituted;
R^{c} is H; and
R^{d} is H.

### Embodiment 7

The method of any one of embodiments 2-6, wherein Aryl² is:

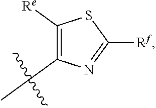

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

### Embodiment 8

The method of embodiment 7, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

### Embodiment 9

The method of embodiment 7 or 8, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

### Embodiment 10

The method of any one of embodiments 7-9, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

### Embodiment 11

The method of any one of embodiments 7-9, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is alkyl.

### Embodiment 12

The method of any one of embodiments 1-10, wherein the compound is:

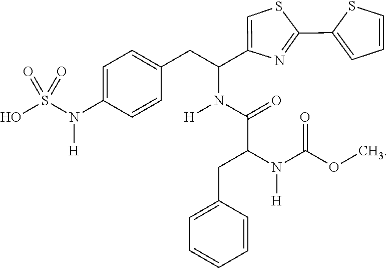 [SMILES: COC(=O)NC(Cc1ccccc1)C(=O)NC(Cc1ccc(NS(=O)(=O)O)cc1)c1csc(-c2cccs2)n1]

### Embodiment 13

The method of any one of embodiments 1-10 and 12, wherein the compound is:

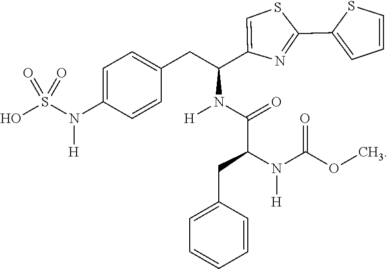 [SMILES: COC(=O)N[C@@H](Cc1ccccc1)C(=O)N[C@@H](Cc1ccc(NS(=O)(=O)O)cc1)c1csc(-c2cccs2)n1]

### Embodiment 14

The method of any one of embodiments 1-9 and 11, wherein the compound is:

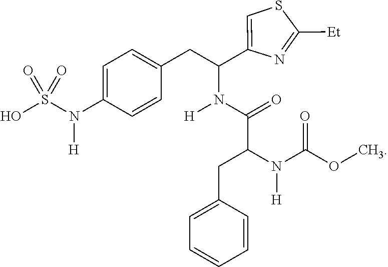 [SMILES: CCc1nc(C(Cc2ccc(NS(=O)(=O)O)cc2)NC(=O)C(Cc2ccccc2)NC(=O)OC)cs1]

### Embodiment 15

The method of any one of embodiments 1-9, 11, and 12, wherein the compound is:

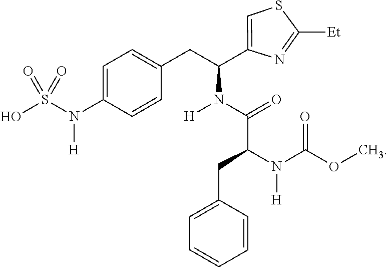 [SMILES: CCc1nc([C@H](Cc2ccc(NS(=O)(=O)O)cc2)NC(=O)[C@H](Cc2ccccc2)NC(=O)OC)cs1]

### Embodiment 16

The method of any one of embodiments 2-6, wherein Aryl² is:

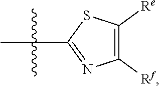

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

### Embodiment 17

The method of embodiment 16, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

### Embodiment 18

The method of embodiment 16 or 17, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

### Embodiment 19

The method of any one of embodiments 16-18, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

### Embodiment 20

The method of any one of embodiments 1-6 and 16-19, wherein the compound is:

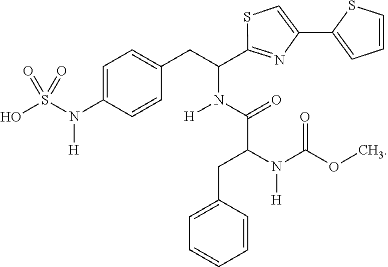 [SMILES: COC(=O)NC(Cc1ccccc1)C(=O)NC(Cc1ccc(NS(=O)(=O)O)cc1)c1nc(-c2cccs2)cs1]

### Embodiment 21

The method of any one of embodiments 1-6 and 16-20, wherein the compound is:

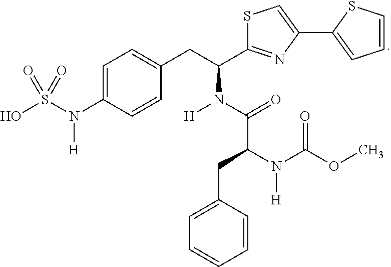 [SMILES: COC(=O)N[C@@H](Cc1ccccc1)C(=O)N[C@@H](Cc1ccc(NS(=O)(=O)O)cc1)c1nc(-c2cccs2)cs1]

### Embodiment 22

The method of any one of embodiments 1-21, wherein the method further comprises administering an agent that improves the aqueous solubility of the compound that activates Tie-2.

### Embodiment 23

The method of any one of embodiments 1-22, wherein the agent that improves the aqueous solubility of the compound that activates Tie-2 comprises a cyclodextrin moiety.

### Embodiment 24

The method of any one of embodiments 1-23, wherein the agent that improves the aqueous solubility of the compound that activates Tie-2 comprises a 2-hydroxypropyl-β-cyclodextrin moiety.

### Embodiment 25

The method of any one of embodiments 1-23, wherein the agent that improves the aqueous solubility of the compound that activates Tie-2 comprises a sulfobutylether-β-cyclodextrin moiety.

### Embodiment 26

The method of any one of embodiments 1-25, wherein the nephropathy is diabetic nephropathy.

### Embodiment 27

The method of any one of embodiments 1-26, wherein the diabetic nephropathy results from hyperglycemia, kidney hyperfiltration, renal injury, glycation products, or cytokine activation.

### Embodiment 28

The method of any one of embodiments 1-27, wherein the therapeutically-effective amount is from about 0.1 mg to about 100 mg.

### Embodiment 29

The method of any one of embodiments 1-28, wherein the therapeutically-effective amount is from about 0.5 mg to about 30 mg.

### Embodiment 30

The method of any one of embodiments 1-29, wherein the compound is administered subcutaneously.

### Embodiment 31

The method of any one of embodiments 1-30, wherein the administering increases a percentage change in a geometric mean of a urine albumin-to-creatinine ratio in the subject.

### Embodiment 32

The method of any one of embodiments 1-31, wherein the administering reduces a urine albumin-to-creatinine ratio in the subject.

### Embodiment 33

A method of treating nephropathy in a subject in need thereof, the method comprising administering to the subject a therapeutically-effective amount of a compound that inhibits HPTPβ.

### Embodiment 34

The method of embodiment 33, wherein the compound has the formula:

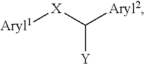

wherein:
Aryl¹ is an aryl group which is substituted or unsubstituted;
Aryl² is an aryl group which is substituted or unsubstituted;
X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

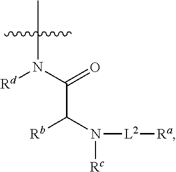

wherein:
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{c} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted,
   or a pharmaceutically-acceptable salt thereof, tautomer, or zwitterion thereof.

### Embodiment 35

The method of embodiment 34, wherein:
Aryl¹ is substituted or unsubstituted phenyl;
Aryl² is substituted or unsubstituted heteroaryl; and
X is alkylene.

### Embodiment 36

The method of embodiment 34 or 35, wherein:
Aryl¹ is substituted phenyl;
Aryl² is substituted heteroaryl; and
X is methylene.

### Embodiment 37

The method of any one of embodiments 34-36, wherein the compound that inhibits HPTPβ is a compound of the formula:

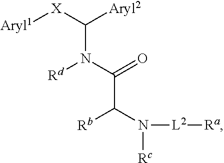

wherein

Aryl¹ is para-substituted phenyl;
Aryl² is substituted heteroaryl;
X is methylene;
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond;
R^{a} is H, alkyl, alkenyl, alkynyl, aryl, arylalkyl, heterocyclyl, heterocyclylalkyl, heteroaryl, or heteroarylalkyl, any of which is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted;
R^{c} is H or alkyl which is substituted or unsubstituted; and
R^{d} is H or alkyl which is substituted or unsubstituted.

### Embodiment 38

The method of any one of embodiments 34-37, wherein:
Aryl² is a substituted thiazole moiety;
L² together with the nitrogen atom to which L² is bound forms a carbamate linkage;
R^{a} is alkyl, which is substituted or unsubstituted;
R^{b} is arylalkyl, which is substituted or unsubstituted;
R^{c} is H; and
R^{d} is H.

### Embodiment 39

The method of any one of embodiments 34-38, wherein Aryl² is:

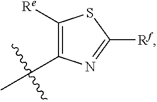

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

### Embodiment 40

The method of embodiment 39, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

### Embodiment 41

The method of embodiment 39 or 40, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

### Embodiment 42

The method of any one of embodiments 39-41, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

### Embodiment 43

The method of any one of embodiments 39-41, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is alkyl.

### Embodiment 44

The method of any one of embodiments 33-42, wherein the compound is:

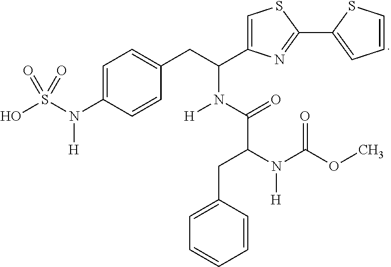 [SMILES: COC(=O)NC(Cc1ccccc1)C(=O)NC(Cc1ccc(NS(=O)(=O)O)cc1)c1csc(-c2cccs2)n1]

### Embodiment 45

The method of any one of embodiments 33-42 and 44, wherein the compound is:

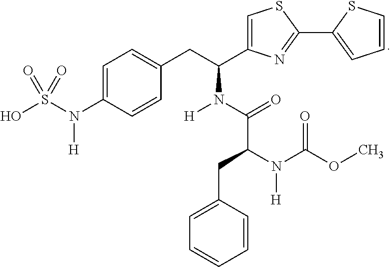 [SMILES: COC(=O)N[C@@H](Cc1ccccc1)C(=O)N[C@@H](Cc1ccc(NS(=O)(=O)O)cc1)c1csc(-c2cccs2)n1]

### Embodiment 46

The method of any one of embodiments 33-41 and 43, wherein the compound is:

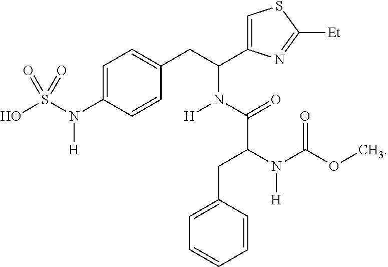 [SMILES: CCc1nc(C(Cc2ccc(NS(=O)(=O)O)cc2)NC(=O)C(Cc2ccccc2)NC(=O)OC)cs1]

### Embodiment 47

The method of any one of embodiments 33-41, 43, and 46, wherein the compound is:

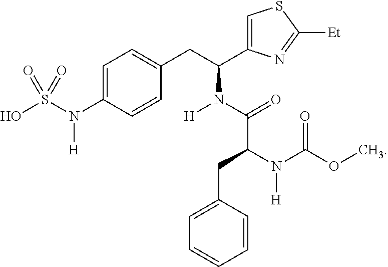 [SMILES: CCc1nc([C@H](Cc2ccc(NS(=O)(=O)O)cc2)NC(=O)[C@H](Cc2ccccc2)NC(=O)OC)cs1]

### Embodiment 48

The method of any one of embodiments 34-38, wherein Aryl² is:

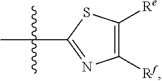

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

### Embodiment 49

The method of embodiment 48, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

### Embodiment 50

The method of embodiment 48 or 49, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

### Embodiment 51

The method of any one of embodiments 48-50, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

### Embodiment 52

The method of any one of embodiments 33-38 and 48-51, wherein the compound is:

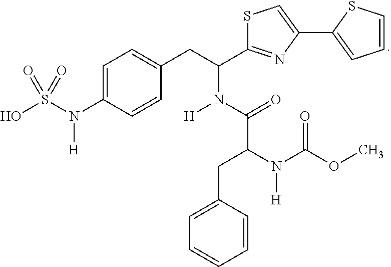 [SMILES: COC(=O)NC(Cc1ccccc1)C(=O)NC(Cc1ccc(NS(=O)(=O)O)cc1)c1nc(-c2cccs2)cs1]

### Embodiment 53

The method of any one of embodiments 33-38 or 48-52, wherein the compound is:

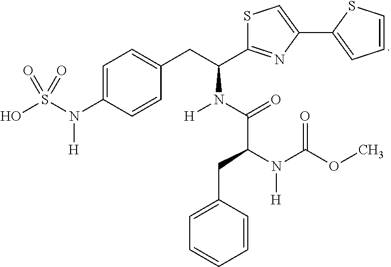 [SMILES: COC(=O)N[C@@H](Cc1ccccc1)C(=O)N[C@@H](Cc1ccc(NS(=O)(=O)O)cc1)c1nc(-c2cccs2)cs1]

### Embodiment 54

The method of any one of embodiments 33-53, wherein the method further comprises administering an agent that improves the aqueous solubility of the compound that inhibits HPTPβ.

### Embodiment 55

The method of any one of embodiments 33-54, wherein the agent that improves the aqueous solubility of the compound that inhibits HPTPβ comprises a cyclodextrin moiety.

### Embodiment 56

The method of any one of embodiments 33-55, wherein the agent that improves the aqueous solubility of the compound that inhibits HPTPβ comprises a 2-hydroxypropyl-β-cyclodextrin moiety.

### Embodiment 57

The method of any one of embodiments 33-55, wherein the agent that improves the aqueous solubility of the compound that inhibits HPTPβ comprises a sulfobutylether-β-cyclodextrin moiety.

### Embodiment 58

The method of any one of embodiments 33-57, wherein the nephropathy is diabetic nephropathy.

### Embodiment 59

The method of any one of embodiments 33-58, wherein the diabetic nephropathy results from hyperglycemia, kidney hyperfiltration, renal injury, glycation products, or cytokine activation.

### Embodiment 60

The method of any one of embodiments 33-59, wherein the therapeutically-effective amount is from about 0.1 mg to about 100 mg.

### Embodiment 61

The method of any one of embodiments 33-60, wherein the therapeutically-effective amount is from about 0.5 mg to about 30 mg.

### Embodiment 62

The method of any one of embodiments 33-61, wherein the compound is administered subcutaneously.

### Embodiment 63

The method of any one of embodiments 33-62, wherein the administering increases a percentage change in a geometric mean of a urine albumin-to-creatinine ratio in the subject.

### Embodiment 64

The method of any one of embodiments 33-63, wherein the administering reduces a urine albumin-to-creatinine ratio in the subject.

### Embodiment 65

A method of treating acute kidney injury in a subject in need thereof, the method comprising administering to the subject a therapeutically-effective amount of a compound that activates Tie-2, wherein the therapeutically-effective amount is about 0.1 mg to about 100 mg.

### Embodiment 66

The method of embodiment 66, wherein the compound has the formula:

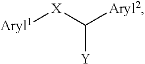

wherein:
Aryl¹ is an aryl group which is substituted or unsubstituted;
Aryl² is an aryl group which is substituted or unsubstituted;
X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

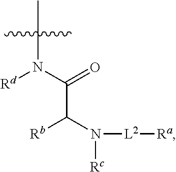

wherein:
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{c} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted,
   or a pharmaceutically-acceptable salt thereof, tautomer, or zwitterion thereof.

### Embodiment 67

The method of embodiment 66, wherein:
Aryl¹ is substituted or unsubstituted phenyl;
Aryl² is substituted or unsubstituted heteroaryl; and
X is alkylene.

### Embodiment 68

The method of embodiment 66 or 67, wherein:
Aryl¹ is substituted phenyl;
Aryl² is substituted heteroaryl; and
X is methylene.

### Embodiment 69

The method of any one of embodiments 66-68, wherein the compound that activates Tie-2 is a compound of the formula:

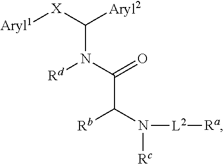

wherein
Aryl¹ is para-substituted phenyl;
Aryl² is substituted heteroaryl;
X is methylene;
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond;
R^{a} is H, alkyl, alkenyl, alkynyl, aryl, arylalkyl, heterocyclyl, heterocyclylalkyl, heteroaryl, or heteroarylalkyl, any of which is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted;
R^{c} is H or alkyl which is substituted or unsubstituted; and
R^{d} is H or alkyl which is substituted or unsubstituted.

### Embodiment 70

The method of any one of embodiments 66-69, wherein:
Aryl² is a substituted thiazole moiety;
L² together with the nitrogen atom to which L² is bound forms a carbamate linkage;
R^{a} is alkyl, which is substituted or unsubstituted;
R^{b} is arylalkyl, which is substituted or unsubstituted;
R^{c} is H; and
R^{d} is H.

### Embodiment 71

The method of any one of embodiments 66-70, wherein Aryl² is:

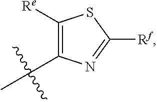

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

### Embodiment 72

The method of embodiment 71, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

### Embodiment 73

The method of embodiment 71 or 72, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

### Embodiment 74

The method of any one of embodiments 71-73, wherein:
Aryl¹ is 4-phenylsulfamic acid;
R^{a} is alkyl, which is substituted or unsubstituted;

R^{b} is arylalkyl, which is substituted or unsubstituted;
R^{e} is H; and
R^{f} is heteroaryl.

### Embodiment 75

The method of any one of embodiments 71-73, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is alkyl.

### Embodiment 76

The method of any one of embodiments 65-74, wherein the compound is:

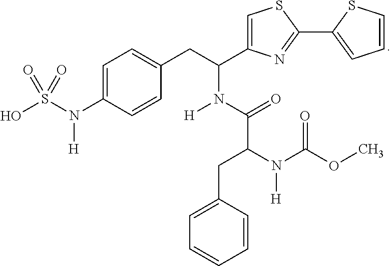 [SMILES: COC(=O)NC(Cc1ccccc1)C(=O)NC(Cc1ccc(NS(=O)(=O)O)cc1)c1csc(-c2cccs2)n1]

### Embodiment 77

The method of any one of embodiments 65-74 and 76, wherein the compound is:

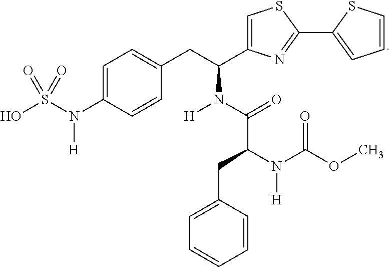 [SMILES: COC(=O)N[C@@H](Cc1ccccc1)C(=O)N[C@@H](Cc1ccc(NS(=O)(=O)O)cc1)c1csc(-c2cccs2)n1]

### Embodiment 78

The method of any one of embodiments 65-73 and 75, wherein the compound is:

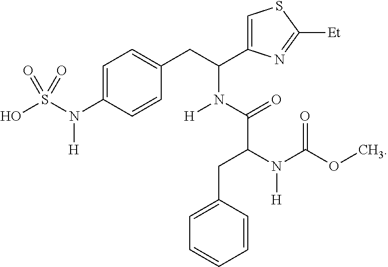 [SMILES: CCc1nc(C(Cc2ccc(NS(=O)(=O)O)cc2)NC(=O)C(Cc2ccccc2)NC(=O)OC)cs1]

### Embodiment 79

The method of any one of embodiments 65-73 and 75, wherein the compound is:

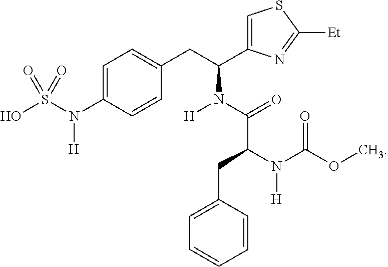 [SMILES: CCc1nc([C@H](Cc2ccc(NS(=O)(=O)O)cc2)NC(=O)[C@H](Cc2ccccc2)NC(=O)OC)cs1]

### Embodiment 80

The method of any one of embodiments 66-70, wherein Aryl² is:

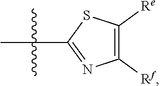

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

### Embodiment 81

The method of embodiment 80, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

### Embodiment 82

The method of embodiment 80 or 81, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

### Embodiment 83

The method of any one of embodiments 80-82, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

### Embodiment 84

The method of any one of embodiments 65-70 and 80-83, wherein the compound is:

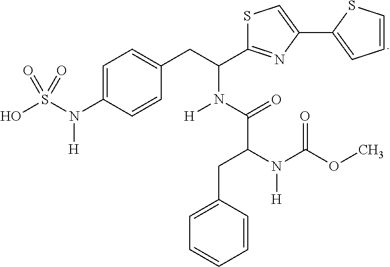 [SMILES: COC(=O)NC(Cc1ccccc1)C(=O)NC(Cc1ccc(NS(=O)(=O)O)cc1)c1nc(-c2cccs2)cs1]

### Embodiment 85

The method of any one of embodiments 65-70 and 80-84, wherein the compound is:

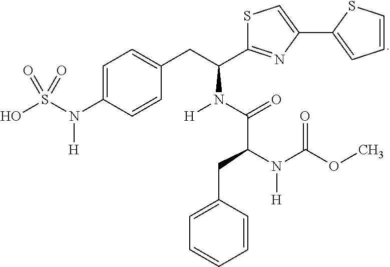 [SMILES: COC(=O)N[C@@H](Cc1ccccc1)C(=O)N[C@@H](Cc1ccc(NS(=O)(=O)O)cc1)c1nc(-c2cccs2)cs1]

### Embodiment 86

The method of any one of embodiments 66-85, wherein the method further comprises administering an agent that improves the aqueous solubility of the compound that activates Tie-2.

### Embodiment 87

The method of any one of embodiments 65-86, wherein the agent that improves the aqueous solubility of the compound that activates Tie-2 comprises a cyclodextrin moiety.

### Embodiment 88

The method of any one of embodiments 65-87, wherein the agent that improves the aqueous solubility of the compound that activates Tie-2 comprises a 2-hydroxypropyl-β-cyclodextrin moiety.

### Embodiment 89

The method of any one of embodiments 65-87, wherein the agent that improves the aqueous solubility of the compound that activates Tie-2 comprises a sulfobutylether-β-cyclodextrin moiety.

### Embodiment 90

The method of any one of embodiments 65-89, wherein the therapeutically-effective amount is from about 0.5 mg to about 30 mg.

### Embodiment 91

The method of any one of embodiments 65-90, wherein the compound is administered subcutaneously.

### Embodiment 92

The method of any one of embodiments 65-91, wherein the administering increases a percentage change in a geometric mean of a urine albumin-to-creatinine ratio in the subject.

### Embodiment 93

The method of any one of embodiments 65-92, wherein the administering reduces a urine albumin-to-creatinine ratio in the subject.

### Embodiment 94

A method of treating acute kidney injury in a subject in need thereof, the method comprising administering to the subject a therapeutically-effective amount of a compound that inhibits HPTPβ, wherein the therapeutically-effective amount is about 0.1 mg to about 100 mg.

### Embodiment 95

The method of embodiment 94, wherein the compound has the formula:

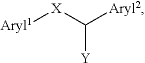

wherein:
Aryl¹ is an aryl group which is substituted or unsubstituted;
Aryl² is an aryl group which is substituted or unsubstituted;
X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

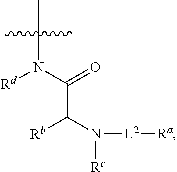

wherein:
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{c} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted,
   or a pharmaceutically-acceptable salt thereof, tautomer, or zwitterion thereof.

### Embodiment 96

The method of embodiment 95, wherein:
Aryl¹ is substituted or unsubstituted phenyl;
Aryl² is substituted or unsubstituted heteroaryl; and
X is alkylene.

### Embodiment 97

The method of embodiment 95 or 96, wherein:
Aryl¹ is substituted phenyl;
Aryl² is substituted heteroaryl; and
X is methylene.

### Embodiment 98

The method of any one of embodiments 95-97, wherein the compound that inhibits HPTPβ is a compound of the formula:

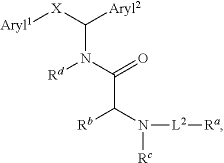

wherein
Aryl¹ is para-substituted phenyl;
Aryl² is substituted heteroaryl;
X is methylene;
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond;
R^{a} is H, alkyl, alkenyl, alkynyl, aryl, arylalkyl, heterocyclyl, heterocyclylalkyl, heteroaryl, or heteroarylalkyl, any of which is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted;
R^{c} is H or alkyl which is substituted or unsubstituted; and
R^{d} is H or alkyl which is substituted or unsubstituted.

### Embodiment 99

The method of any one of embodiments 95-98, wherein:
Aryl² is a substituted thiazole moiety;
L² together with the nitrogen atom to which L² is bound forms a carbamate linkage;
R^{a} is alkyl, which is substituted or unsubstituted;
R^{b} is arylalkyl, which is substituted or unsubstituted;
R^{c} is H; and
R^{d} is H.

### Embodiment 100

The method of any one of embodiments 95-99, wherein Aryl² is:

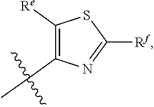

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

### Embodiment 101

The method of embodiment 100, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

### Embodiment 102

The method of embodiment 100 or 101 wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

### Embodiment 103

The method of any one of embodiments 100-102, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

### Embodiment 104

The method of any one of embodiments 100-102, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is alkyl.

### Embodiment 105

The method of any one of embodiments 94-103, wherein the compound is:

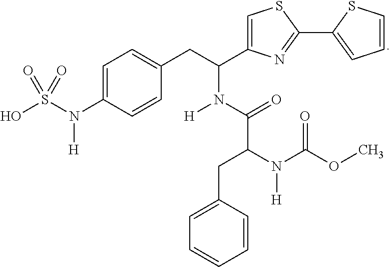 [SMILES: COC(=O)NC(Cc1ccccc1)C(=O)NC(Cc1ccc(NS(=O)(=O)O)cc1)c1csc(-c2cccs2)n1]

### Embodiment 106

The method of any one of embodiments 94-103 and 105, wherein the compound is:

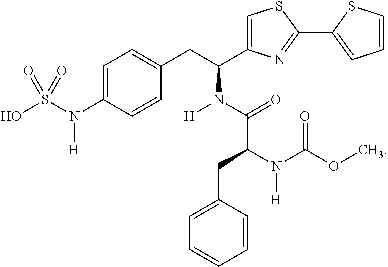 [SMILES: COC(=O)N[C@@H](Cc1ccccc1)C(=O)N[C@@H](Cc1ccc(NS(=O)(=O)O)cc1)c1csc(-c2cccs2)n1]

### Embodiment 107

The method of any one of embodiments 94-102 and 104, wherein the compound is:

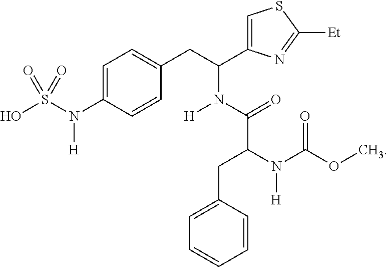 [SMILES: CCc1nc(C(Cc2ccc(NS(=O)(=O)O)cc2)NC(=O)C(Cc2ccccc2)NC(=O)OC)cs1]

### Embodiment 108

The method of any one of embodiments 94-102, 104, and 107, wherein the compound is:

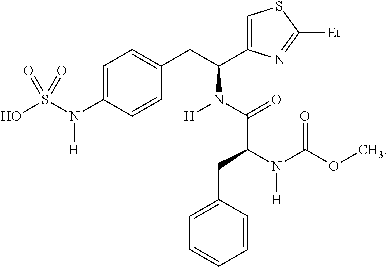 [SMILES: CCc1nc([C@H](Cc2ccc(NS(=O)(=O)O)cc2)NC(=O)[C@H](Cc2ccccc2)NC(=O)OC)cs1]

### Embodiment 109

The method of any one of embodiments 95-99, wherein Aryl² is:

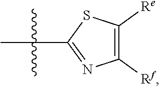

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

### Embodiment 110

The method of embodiment 109, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

### Embodiment 111

The method of embodiment 109 or 110, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

### Embodiment 112

The method of any one of embodiments 109-111, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

### Embodiment 113

The method of any one of embodiments 94-99 and 109-112, wherein the compound is:

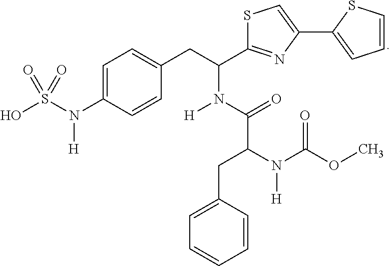 [SMILES: COC(=O)NC(Cc1ccccc1)C(=O)NC(Cc1ccc(NS(=O)(=O)O)cc1)c1nc(-c2cccs2)cs1]

### Embodiment 114

The method of any one of embodiments 94-99 and 109-113, wherein the compound is:

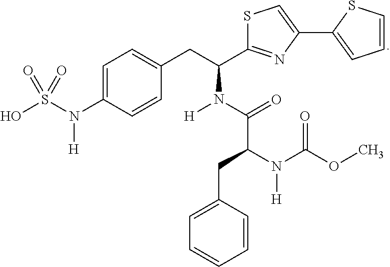 [SMILES: COC(=O)N[C@@H](Cc1ccccc1)C(=O)N[C@@H](Cc1ccc(NS(=O)(=O)O)cc1)c1nc(-c2cccs2)cs1]

### Embodiment 115

The method of any one of embodiments 94-114, wherein the method further comprises administering an agent that improves the aqueous solubility of the compound that inhibits HPTPβ.

### Embodiment 116

The method of any one of embodiments 94-115, wherein the agent that improves the aqueous solubility of the compound that inhibits HPTPβ comprises a cyclodextrin moiety.

### Embodiment 117

The method of any one of embodiments 94-116, wherein the agent that improves the aqueous solubility of the compound that inhibits HPTPβ comprises a 2-hydroxypropyl-β-cyclodextrin moiety.

### Embodiment 118

The method of any one of embodiments 94-116, wherein the agent that improves the aqueous solubility of the compound that inhibits HPTPβ comprises a sulfobutylether-β-cyclodextrin moiety.

### Embodiment 119

The method of any one of embodiments 94-118, wherein the therapeutically-effective amount is from about 0.5 mg to about 30 mg.

### Embodiment 120

The method of any one of embodiments 94-119, wherein the compound is administered subcutaneously.

### Embodiment 121

The method of any one of embodiments 94-120, wherein the administering increases a percentage change in a geometric mean of a urine albumin-to-creatinine ratio in the subject.

### Embodiment 123

The method of any one of embodiments 94-121, wherein the administering reduces a urine albumin-to-creatinine ratio in the subject.

What is claimed is:

1. A method of treating nephropathy in a subject in need thereof, the method comprising administering to the subject a therapeutically-effective amount of a compound that activates Tie-2.

2. The method of claim 1, wherein the compound has the formula: 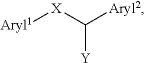
wherein:
Aryl¹ is an aryl group which is substituted or unsubstituted;
Aryl² is an aryl group which is substituted or unsubstituted;
X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or 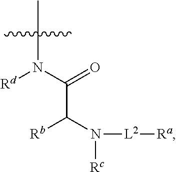
wherein:
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{c} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted,
or a pharmaceutically-acceptable salt thereof, tautomer, or zwitterion thereof.

3. The method of claim 2, wherein:
Aryl¹ is substituted or unsubstituted phenyl;
Aryl² is substituted or unsubstituted heteroaryl; and
X is alkylene.

4. The method of claim 3, wherein:
Aryl¹ is substituted phenyl;
Aryl² is substituted heteroaryl; and
X is methylene.

5. The method of claim 3, wherein the compound that activates Tie-2 is a compound of the formula: 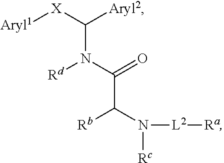
wherein
Aryl¹ is para-substituted phenyl;
Aryl² is substituted heteroaryl;
X is methylene;
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond;
R^{a} is H, alkyl, alkenyl, alkynyl, aryl, arylalkyl, heterocyclyl, heterocyclylalkyl, heteroaryl, or heteroarylalkyl, any of which is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted;
R^{c} is H or alkyl which is substituted or unsubstituted; and
R^{d} is H or alkyl which is substituted or unsubstituted.

6. The method of claim 5, wherein:
Aryl² is a substituted thiazole moiety;
L² together with the nitrogen atom to which L² is bound forms a carbamate linkage;
R^{a} is alkyl, which is substituted or unsubstituted;
R^{b} is arylalkyl, which is substituted or unsubstituted;
R^{c} is H; and
R^{d} is H.

7. The method of claim 6, wherein Aryl² is: 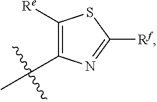
wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

8. The method of claim 7, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

9. The method of claim 8, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

10. The method of claim 9, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

11. The method of claim 7, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is alkyl.

12. The method of claim 2, wherein the compound is:

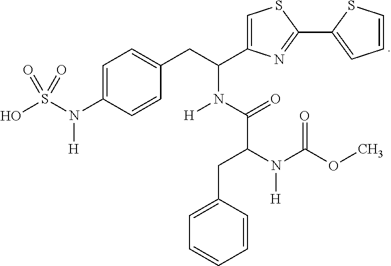 [SMILES: COC(=O)NC(Cc1ccccc1)C(=O)NC(Cc1ccc(NS(=O)(=O)O)cc1)c1csc(-c2cccs2)n1]

13. The method of claim 2, wherein the compound is:

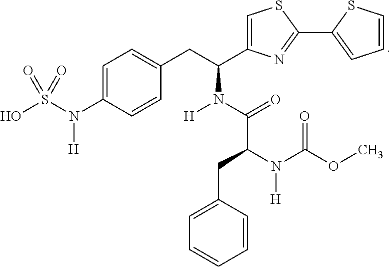 [SMILES: COC(=O)N[C@@H](Cc1ccccc1)C(=O)N[C@@H](Cc1ccc(NS(=O)(=O)O)cc1)c1csc(-c2cccs2)n1]

14. The method of claim 2, wherein the compound is:

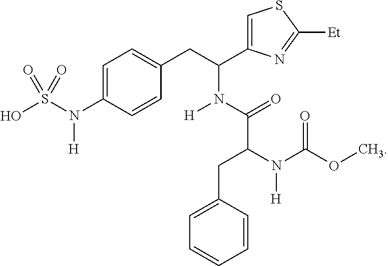 [SMILES: CCc1nc(C(Cc2ccc(NS(=O)(=O)O)cc2)NC(=O)C(Cc2ccccc2)NC(=O)OC)cs1]

15. The method of claim 2, wherein the compound is:

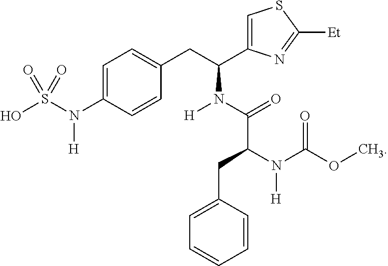 [SMILES: CCc1nc([C@H](Cc2ccc(NS(=O)(=O)O)cc2)NC(=O)[C@H](Cc2ccccc2)NC(=O)OC)cs1]

16. The method of claim 6, wherein Aryl² is: 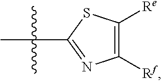
wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

17. The method of claim 16, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

18. The method of claim 17, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

19. The method of claim 18, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

20. The method of claim 2, wherein the compound is:

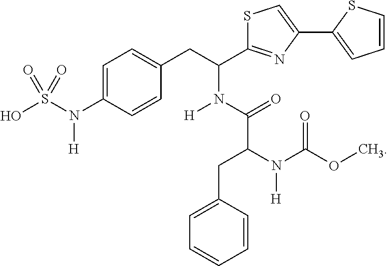 [SMILES: COC(=O)NC(Cc1ccccc1)C(=O)NC(Cc1ccc(NS(=O)(=O)O)cc1)c1nc(-c2cccs2)cs1]

21. The method of claim 2, wherein the compound is:

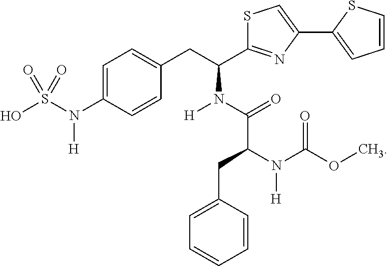 [SMILES: COC(=O)N[C@@H](Cc1ccccc1)C(=O)N[C@@H](Cc1ccc(NS(=O)(=O)O)cc1)c1nc(-c2cccs2)cs1]

22. The method of claim 1, wherein the nephropathy is diabetic nephropathy.

23. The method of claim 1, wherein the therapeutically-effective amount is from about 0.1 mg to about 100 mg.

24. The method of claim 23, wherein the therapeutically-effective amount is from about 0.5 mg to about 30 mg.

25. The method of claim 1, wherein the compound is administered subcutaneously.

26. The method of claim 1, wherein the administering reduces a urine albumin-to-creatinine ratio by at least about 20% in the subject.
